(19) United States
(12) Patent Application Publication
(10) Pub. No.: US 2022/0177534 A1
(43) Pub. Date: Jun. 9, 2022

(54) CCL21 AND CHECKPOINT INHIBITORS FOR THE TREATMENT OF CANCER

(71) Applicants: The Regents of the University of California, Oakland, CA (US); The United States Government Represented by the Department of Veterans Affairs, Washington, DC (US)

(72) Inventors: Steven M. DUBINETT, Los Angeles, CA (US); Jay Moon LEE, Manhattan Beach, CA (US); Bin LIU, Woodland Hills, CA (US); Ramin SALEHI-RAD, Encino, CA (US); Sharvendra SHARMA, Culver City, CA (US)

(73) Assignees: The Regents of the University of California, Oakland, CA (US); The United States Government Represented by the Department of Veterans Affairs, Washington, DC (US)

(21) Appl. No.: 17/053,872

(22) PCT Filed: May 10, 2019
(86) PCT No.: PCT/US19/31834

§ 371 Date: Jun. 17, 2021

Related U.S. Application Data
(60) Provisional application No. 62/828,352, filed on Apr. 2, 2019.
(60) Provisional application No. 62/669,707, filed on May 10, 2018.

(51) Int. Cl. C07K 14/52; C07K 14/705

(52) U.S. Cl. CPC C07K 14/521; A61K 38/00; C07K 14/70596

(57) ABSTRACT

The present disclosure relates, in general, to methods for treating cancer comprising administering to a subject in need thereof an effective amount of dendritic cells comprising the human CCL21 gene in combination with an anti-PD-1 antibody. In one aspect, the treatment is amenable to patients with tumors having a high mutational burden.

### CROSS-REFERENCE TO RELATED APPLICATIONS

This application is a National Phase Application of PCT International Application No. PCT/US2019/031834, International Filing Date May 10, 2019, claiming the benefit of U.S. Patent Applications Nos. 62/669,707, filed May 10, 2018, and 62/828,352, filed Apr. 2, 2019, which are hereby incorporated by reference.

### GOVERNMENT SUPPORT STATEMENT

This invention was made with government support under Grant Number CA105705, awarded by the National Institutes of Health. The government has certain rights in the invention. This work was supported by the U.S. Department of Veterans Affairs, and the Federal Government has certain rights in the invention.

### FIELD OF THE DISCLOSURE

The present disclosure relates, in general, to methods of treating cancer comprising administering autologous antigen presenting cells expressing CCL21 in combination with checkpoint inhibitors, such as anti-PD-1 antibody, to reduce cancer growth and other symptoms in a subject.

### BACKGROUND OF THE DISCLOSURE

Lung cancer is the most common cause of cancer deaths in both the United States and the world (1, 2). In the United States alone, approximately 200,000 people are diagnosed with lung cancer each year. Almost 85% of patients with lung cancer will die of the disease within 5 years of diagnosis (3). The high case-fatality rate is caused by two overriding issues. First, patients are frequently diagnosed with advanced stage disease. Second, even among patients diagnosed with earlier stage disease and treated, recurrence is common. As a result, the majority of patients diagnosed with lung cancer will have metastatic disease at some point in their course.

Over 85% of patients with lung cancer have non-small cell lung cancer (NSCLC), which is the group of lung cancer histologies (adenocarcinoma, squamous cell carcinoma and large cell carcinoma)(4). Metastatic NSCLC has a poor prognosis, with the majority of patients failing to achieve an objective response to chemotherapy (13). Chemotherapy also is often associated with an unfavorable side effect profile. Statistics from American Cancer Society reveal that the five-year survival for metastatic NSCLC is approximately 1%. For patients whose tumors harbor activating mutations in EGFR or ALK fusion, the options have improved over the past decade, but prior to the availability of inhibitors of the PD-1/PD-L1 axis, little improvement was seen for the 85% of patients without these molecular abnormalities.

Although inhibition of the PD-1/PD-L1 axis has proven to be a very effective approach in some patients with NSCLC, the majority of patients do not benefit from the current immunotherapeutic approaches. The initial evaluation of PD-1 inhibitors included patients who had previously received standard platinum-based chemotherapy (12, 14-18). Studies of a variety of agents, including the PD-1 inhibitors nivolumab and pembrolizumab and the PD-L1 inhibitor atezolizumab, show objective responses in approximately 20% of patients. Durable responses are often seen.

In the KEYNOTE-024 trial, patients with metastatic NSCLC who had not had prior chemotherapy, and who had PD-L1 staining in at least half of their tumor cells, were randomized to pembrolizumab or platinum-based chemotherapy (5). Nearly half of the patients achieved an objective response with pembrolizumab; and pembrolizumab led to a longer progression-free survival and overall survival than chemotherapy.

In contrast to the KEYNOTE-024 trial which was restricted to patients with PD-L1 expression in at least half of their cells, a study of similar design with the PD-1 inhibitor nivolumab, CheckMate 026, included patients with lower levels of staining for PD-L1 (the primary analysis was in patients with staining in at least 5% of their tumor cells) (19). The CheckMate 026 study did not show a benefit for the primary endpoint of progression free survival, with both the median and hazard ratio numerically favoring chemotherapy. Thus, there remains a need for immunotherapeutic approaches for the initial treatment of metastatic NSCLC in patients who do not have PD-L1 expression in at least half of their tumor cells. This includes the majority of patients with metastatic disease.

Patients with stage IV NSCLC and less than 50% staining for PD-L1 currently receive platinum-based chemotherapy. The shortcomings of chemotherapy are well known. The only approved treatment option, apart from platinum-based chemotherapy alone, is the combination of this chemotherapy, i.e. carboplatin and pemetrexed, with pembrolizumab. Based on the histology-specific approval for pemetrexed in patients with non-squamous NSCLC, this combination is approved only in non-squamous disease. This accelerated approval will require the completion of large randomized trials for full approval.

Previously, many oncologists had not embraced the combination of pembrolizumab and chemotherapy. This combination has been shown to increase the toxicity experienced by the patient as compared to either treatment alone. The study that led to the approval of this approach included only 123 patients, and had a favorable outcome compared to other studies evaluating chemotherapy with a PD-1 or PD-L1 inhibitor (20). Further, the study was not designed to robustly assess survival, and therefore an open question remains regarding the value of this combination in light of the associated toxicities. Recently, an application for approval of this combination in Europe was withdrawn. A recent study, however, reported on a phase III trial indicating that first-line pembrolizumab plus chemotherapy (pemetrexed plus cisplatin or carboplatin) in patients with advanced or metastatic NSCLC, irrespective of PD-L1 expression, reduced risk of death by 51% at median follow up (10.5 months) compared to patients receiving doublet chemotherapy (Gandhi et al: Pembrolizumab plus Chemotherapy in Metastatic Non-Small-Cell Lung Cancer. N Engl J Med 2018 May 31; 378(22):2078-2092).

Currently there are no approved immunotherapy combinations for NSCLC, such as combining a PD-1 or PD-L1 inhibitor with a second drug designed to increase the immune response. The combination that has received the most interest is the combination of PD-1/PD-L1 inhibitors and CTLA-4 inhibitors. Nivolumab (anti-PD-1) and ipilimumab (anti-CTLA-4) is an approved combination of this type in melanoma, a disease in which each agent is individually efficacious (21). Results from the CheckMate 227 phase III clinical trial indicate that patients with advanced NSCLC—squamous and non-squamous—and high tumor mutational burden had increased progression-free survival (PFS) when treated with first-line combination nivolumab+ipilimumab compared to chemotherapy, regardless of tumor PD-L1 expression (Hellmann et al: Nivolumab plus Ipilimumab in Lung Cancer with a High Tumor Mutational Burden. N Engl J Med 2018 May 31; 378(22):2093-2104).

In another randomized study, the MYSTIC study, which is a study of the PD-L1 inhibitor durvalumab and the CTLA-4 inhibitor tremelimumab, a press release states that progression-free survival with the combined therapy was no better than platinum-based chemotherapy in patients with staining for PD-L1 in at least a quarter of their cells.

From the perspective of the patient, the current therapies for metastatic NSCLC are clearly suboptimal (23). Cytotoxic chemotherapy induces significant toxicity with no ability to cure patients with metastatic NSCLC. Durable responses clearly have been seen with inhibitors of the PD-1/PD-L1 axis. However, among the population of patients being evaluated as part of this trial, no more than 15% of patients would be anticipated to achieve an objective response, and durable responses would be expected in even fewer. The potential of durable clinical benefit in a setting in which such an outcome is currently unlikely, particularly in light of the generally favorable side effect profile of immunotherapeutic approaches, makes this approach quite appealing for the population of patients who would be eligible for the trial.

International application PCT/US2015/059297, published as WO2016/073759, is incorporated herein by reference in its entirety.

### SUMMARY OF THE DISCLOSURE

The present disclosure provides an improved method for treating cancer and reducing progression of tumors in a subject comprising administering secondary lymphoid tissue cytokine (SLC) polypeptide, also called CCL21, or cells expressing the human CCL21 gene in combination with a checkpoint inhibitor. It is contemplated that the combination of administration will improve the efficacy of administering the checkpoint inhibitor alone, especially in tumor populations in which less than 50% of the tumor cells express the PD-L1 protein.

In various embodiments, the disclosure provides a method for treating cancer or reducing the reoccurrence of cancer in a subject in need thereof comprising administering an effective amount of a combination therapy comprising a) SLC (CCL21) polypeptide or dendritic cells comprising an vector-CCL21 (vector-CCL21-DC) construct on days 0, 21, and 42, and b) an effective amount of anti-PD-1 antibody every three weeks starting on day 0.

In various embodiments, the vector is a viral vector, a liposome, or other delivery vector. In various embodiments, the viral vector is an adenovirus, an adeno-associated virus, a lentivirus, a retrovirus, a vaccinia virus, modified Ankara virus, sindbis virus, herpesvirus, CMV, or vesicular stomatitis virus. In various embodiments, the adeno-associated virus (AAV) is selected from the group consisting of AAV1, AAV2, AAV3, AAV4, AAV5, AAV6, AAV7, AAV8 or combinations thereof.

In various embodiments, the disclosure provides a method for treating cancer or reducing the reoccurrence of cancer in a subject in need thereof comprising administering an effective amount of a combination therapy comprising a) dendritic cells comprising an Adenovirus-CCL21 (Ad-CCL21-DC) construct on days 0, 21, and 42, and b) an effective amount of anti-PD-1 antibody every three weeks starting on day 0.

In various embodiments, the disclosure provides a method for treating lung cancer or reducing the reoccurrence of lung cancer in a subject in need thereof comprising administering an effective amount of a combination therapy comprising a) dendritic cells comprising an Adenovirus-CCL21 (Ad-CCL21-DC) construct on days 0, 21, and 42, and b) an effective amount of anti-PD-1 antibody every three weeks starting on day 0. In various embodiments, the anti-PD-1 antibody is selected from the group consisting of Pembrolizumab, Nivolumab, Pidilizumab, and Lambrolizumab. In various embodiments, the anti-PD-1 antibody is pembrolizumab administered at a dose of 200 mg every three weeks.

In various embodiments, the Ad-CCL21-DC administered in a dose from 5×106 cells/injection to 3×107 cells/injection. In some embodiments, the Ad-CCL21-DC dose is 5×106, 1×107, or 3×107 cells/injection.

In various embodiments, the cancer is a solid tumor. Exemplary cancers contemplated in the method are described more fully in the Detailed Description.

In various embodiments, the cancer expresses PD-L1 in less than 50% of tumor cells. In various embodiments, the cancer expresses PD-L1 in 50% or greater of the tumor cells. In one embodiment, expression of PD-L1 on tumor cells is assessed by immunohistochemical staining of the cells. In various embodiments, the subject has received first line pembrolizumab plus chemotherapy, or, alternatively, the subject has failed initial therapy with this combination.

In various embodiments, the cancer is lung cancer. In various embodiments, the lung cancer is non-small cell lung carcinoma (NSCLC). In various embodiments, the lung cancer is stage IV NSCLC expressing PD-L1 in less than 50% of cells.

In various embodiments, the NSCLC or other solid tumor is a squamous cell or non-squamous cell tumor. In various embodiments, the subject has a high tumor mutational burden. Tumor mutational burden may be monitored by diagnostic assay, e.g., from FoundationOne (Cambridge, Mass.), such as FoundationOne CDx™, FoundationOne®, FoundationAct®, or FoundationOne®Heme.

In various embodiments, the patient has a NSCLC tumor accessible by CT-guided intervention or bronchoscopy, and the patient is naïve to systemic treatment for NSCLC. In various embodiments, the vector-CCL21-DC is administered via CT-guided or bronchoscopic IT injection.

In various embodiments, the anti-PD-1 antibody is administered intravenously.

In various embodiments, the vector-CCL21-DC increases CD8 T cell infiltration into a tumor. In various embodiments, the CD8 cells are increased by 2-fold or more in the treated subject compared to a subject not receiving combination therapy.

In various embodiments, the vector-CCL21-DC increases PD-L1 expression in a tumor.

In various embodiments, the dendritic cells are autologous cells from the patient.

In various embodiments, the CCL21 comprises an amino acid sequence of SEQ ID NO: 1 or SEQ ID NO: 2.

In various embodiments, the adenovirus is a replication-deficient adenoviral vector.

In various embodiments, the tumor volume is decreased by 20% or more. In various embodiments, tumor size in the subject is decreased by about 25-50%, about 40-70% or about 50-90% or more. In various embodiments, the therapy reduces the rate of metastasis and/or slows progression of the tumor in the patient.

In various embodiments, the anti-PD-1 antibody is administered within 1 hour after vector-CCL21-DC therapy on days 0, 21 and 42.

In various embodiments, the combination therapy increases the number of tumor antigen specific CD8 and CD4 cells in the subject.

In various embodiments, a method of treating cancer or a solid tumor having a high mutational burden in a subject is provided comprising a. administering to the subject (i) a SLC polypeptide, (ii) a polynucleotide encoding the SLC polypeptide, (iii) a cell comprising a polynucleotide encoding the SLC polypeptide, or (iv) any combination thereof, and b. administering to the subject an immune checkpoint inhibitor.

In various embodiments thereof, the immune checkpoint inhibitor is selected from the group consisting of a CTLA-4 inhibitor, a CTLA-4 receptor inhibitor, a PD-1 inhibitor, a PD-L1 inhibitor, a PD1-L2 inhibitor, a 4-1BB inhibitor, an OX40 inhibitor, a LAG-3 inhibitor, a TIM-3 inhibitor, or a combination thereof.

In various embodiments thereof, the immune checkpoint inhibitor is an antibody, optionally, a monoclonal antibody.

In various embodiments thereof, the immune checkpoint inhibitor is a CTLA-4 inhibitor, optionally, ipilimumab or tremilimumab.

In various embodiments thereof, the immune checkpoint inhibitor is a PDl inhibitor selected from a group consisting of: Nivolumab, Pembrolizumab, Pidilizumab, Lambrolizumab, BMS-936559, Atezolizumab, and AMP-224, AMP224, AUNP12, BGB108, MCLA134, MEDIO680, PDROOI, REGN2810, SHR1210, STIAl lOX, STIAlllO and TSR042.

In various embodiments thereof, the immune checkpoint inhibitor is a PD-Ll inhibitor selected from a group consisting of: BMS-936559, MPDL3280A, MEDI-4736, MSB0010718C, ALN-PDL, BGBA317, KD033, KY1003, STIA100X, STIA1010, STIA1011, STIA1012 and STIA1014.

In various embodiments thereof, the SLC polypeptide comprises an amino acid sequence of SEQ ID NO: 1 or SEQ ID NO: 2.

In various embodiments thereof, the polynucleotide encoding the SLC polypeptide is inserted into a vector and the vector is administered to the subject. In various embodiments thereof, the vector is an adenoviral vector. In various embodiments thereof, the adenoviral vector is a replication-deficient adenoviral vector.

In various embodiments thereof, the cell comprising the polynucleotide encoding the SLC polypeptide is an antigen presenting cell (APC). In various embodiments thereof, the APC is a dendritic cell. In various embodiments thereof, the dendritic cell is autologous to the subject.

In various embodiments thereof, at least or about 1×10⁶ cells comprising the polynucleotide encoding the SLC polypeptide are administered to the subject. In various embodiments thereof, the cells produce at least or about 0.25 ng of CCL21 per 1×10⁶ cells in a 24-hour period.

In various embodiments thereof, the subject comprises a solid tumor and the cells are administered to the subject intratumorally.

In various embodiments thereof, the solid tumor is a non-small cell lung carcinoma (NSCLC) solid tumor.

In various embodiments thereof, the (i) SLC polypeptide, (ii) polynucleotide encoding the SLC polypeptide, (iii) cell comprising the polynucleotide encoding the SLC polypeptide, or (iv) any combination thereof, is administered to the subject prior to immune checkpoint inhibitor.

In various embodiments thereof, the (i) SLC polypeptide, (ii) polynucleotide encoding the SLC polypeptide, (iii) cell comprising a polynucleotide encoding the SLC polypeptide, or (iv) any combination thereof, is administered to the subject about 2 weeks prior to the immune checkpoint inhibitor. In various embodiments thereof, the (i) SLC polypeptide, (ii) polynucleotide encoding the SLC polypeptide, (iii) cell comprising a polynucleotide encoding the SLC polypeptide, or (iv) any combination thereof, is administered to the subject more than once.

In various embodiments thereof, the (i) SLC polypeptide, (ii) polynucleotide encoding the SLC polypeptide, (iii) cell comprising a polynucleotide encoding the SLC polypeptide, or (iv) any combination thereof, is administered to the subject once a month. In various embodiments thereof, the immune checkpoint inhibitor is administered to the subject more than once. In various embodiments thereof, the immune checkpoint inhibitor is administered to the subject once every 2 weeks.

In various embodiments, a method of treating a cancer or a solid tumor in a subject is provided comprising the steps of: a. identifying the presence of a high mutational burden in the tumor of the subject; b. administering to the subject having a high mutational burden tumor (i) a SLC polypeptide, (ii) a polynucleotide encoding the SLC polypeptide, (iii) a cell comprising a polynucleotide encoding the SLC polypeptide, or (iv) any combination thereof, and c. administering to the subject an immune checkpoint inhibitor.

In various embodiments thereof, the immune checkpoint inhibitor is selected from the group consisting of a CTLA-4 inhibitor, a CTLA-4 receptor inhibitor, a PD-1 inhibitor, a PD-L1 inhibitor, a PD1-L2 inhibitor, a 4-1BB inhibitor, an OX40 inhibitor, a LAG-3 inhibitor, a TIM-3 inhibitor, or a combination thereof.

In various embodiments thereof, the immune checkpoint inhibitor is an antibody, optionally, a monoclonal antibody.

In various embodiments thereof, the immune checkpoint inhibitor is a CTLA-4 inhibitor, optionally, ipilimumab or tremilimumab.

In various embodiments thereof, the immune checkpoint inhibitor is a PDl inhibitor selected from a group consisting of: Nivolumab, Pembrolizumab, Pidilizumab, Lambrolizumab, BMS-936559, Atezolizumab, and AMP-224, AMP224, AUNP12, BGB108, MCLA134, MEDIO680, PDROOI, REGN2810, SHR1210, STIA1 lOX, STIAl llO and TSR042.

In various embodiments thereof, the immune checkpoint inhibitor is a PD-Ll inhibitor selected from a group consisting of: BMS-936559, MPDL3280A, MEDI-4736, MSB0010718C, ALN-PDL, BGBA317, KD033, KY1003, STIA100X, STIA1010, STIA1011, STIA1012 and STIA1014.

In various embodiments thereof, the SLC polypeptide comprises an amino acid sequence of SEQ ID NO: 1 or SEQ ID NO: 2.

In various embodiments thereof, the polynucleotide encoding the SLC polypeptide is inserted into a vector and the vector is administered to the subject. In various embodiments thereof, the vector is an adenoviral vector. In various embodiments thereof, the adenoviral vector is a replication-deficient adenoviral vector.

In various embodiments thereof, the cell comprising the polynucleotide encoding the SLC polypeptide is an antigen presenting cell (APC). In various embodiments thereof, the APC is a dendritic cell. In various embodiments thereof, the dendritic cell is autologous to the subject.

In various embodiments thereof, at least or about 1×10⁶ cells comprising the polynucleotide encoding the SLC polypeptide are administered to the subject. In various embodiments thereof, the cells produce at least or about 0.25 ng of CCL21 per 1×10⁶ cells in a 24-hour period.

In various embodiments thereof, the subject comprises a solid tumor and the cells are administered to the subject intratumorally.

In various embodiments thereof, the solid tumor is a non-small cell lung carcinoma (NSCLC) solid tumor.

In various embodiments thereof, the (i) SLC polypeptide, (ii) polynucleotide encoding the SLC polypeptide, (iii) cell comprising a polynucleotide encoding the SLC polypeptide, or (iv) any combination thereof, is administered to the subject prior to immune checkpoint inhibitor. In various embodiments thereof, the (i) SLC polypeptide, (ii) polynucleotide encoding the SLC polypeptide, (iii) cell comprising a polynucleotide encoding the SLC polypeptide, or (iv) any combination thereof, is administered to the subject about 2 weeks prior to the immune checkpoint inhibitor.

In various embodiments thereof, the (i) SLC polypeptide, (ii) polynucleotide encoding the SLC polypeptide, (iii) cell comprising a polynucleotide encoding the SLC polypeptide, or (iv) any combination thereof, is administered to the subject more than once. In various embodiments thereof, the (i) SLC polypeptide, (ii) polynucleotide encoding the SLC polypeptide, (iii) cell comprising a polynucleotide encoding the SLC polypeptide, or (iv) any combination thereof, is administered to the subject once a month.

In various embodiments thereof, the immune checkpoint inhibitor is administered to the subject more than once. In various embodiments thereof, the immune checkpoint inhibitor is administered to the subject once every 2 weeks.

In any of the foregoing embodiments thereof, the high mutational burden is determined by a biopsy of the tumor. In various embodiments thereof, tumor-associated neoantigens are determined. In various embodiments thereof, the efficacy of combination therapy is followed by elucidation of the neoantigen landscape of the tumor.

In various embodiments thereof, the tumor comprises a mutation selected from KRAS, TP53 (KP) or STK11/LKB1, or any combination thereof. In various embodiments thereof, the tumor has intratumoral heterogeneity.

In various embodiments thereof, the tumor mutational burden is determined by diagnostic assay selected from FoundationOne CDx™, FoundationOne®, FoundationAct®, and FoundationOne®Heme.

In various embodiments thereof, the tumor does not have an activating mutation in the epidermal growth factor receptor or an anaplastic lymphoma kinase gene (ALK) fusion.

In various embodiments thereof, the somatic mutational load and tumor-associated neoantigens before, during and after treatment are used to initiate, prescribe and monitor therapy.

In various embodiments, a method for treating a high mutational burden cancer or reducing the reccurrence of a high mutational burden cancer in a subject in need thereof is provided, comprising administering an effective amount of a combination therapy comprising a) dendritic cells comprising an vector-CCL21 (vector-CCL21-DC) construct on days 0, 21, and 42, and b) an effective amount of anti-PD-1 antibody every three weeks starting on day 0, optionally wherein the vector is an adenoviral vector (Ad-CCL21-DC).

In various embodiments thereof, the anti-PD-1 antibody is selected from the group consisting of Nivolumab, Pembrolizumab, Pidilizumab, and Lambrolizumab.

In various embodiments thereof, the anti-PD-1 antibody is pembrolizumab administered at a dose of 200 mg every three weeks.

In various embodiments thereof, the Ad-CCL21-DC administered in a dose from 5×106 cells/injection to 3×107 cells/injection.

In various embodiments thereof, the Ad-CCL21-DC dose is 5×106, 1×107, or 3×107 cells/injection.

In various embodiments thereof, the lung cancer is stage IV NSCLC expressing PD-L1 in less than 50% of cells.

In various embodiments thereof, the patient has a NSCLC tumor accessible by CT-guided intervention or bronchoscopy, and the patient is naïve to systemic treatment for NSCLC. In various embodiments thereof, the Ad-CCL21-DC is administered via CT-guided or bronchoscopic IT injection.

In various embodiments thereof, the anti-PD-1 antibody is administered intravenously.

In various embodiments thereof, the vector-CCL21-DC increases CD8+ T cell infiltration into a tumor.

In various embodiments thereof, the CD8+ cells are increased by 2-fold or more in the treated subject compared to a subject not receiving combination therapy.

In various embodiments thereof, the vector-CCL21-DC increases PD-L1 expression in a tumor.

In various embodiments thereof, the dendritic cells are autologous cells from the patient.

In various embodiments thereof, the CCL21 comprises an amino acid sequence of SEQ ID NO: 1 or SEQ ID NO:2.

In various embodiments thereof, the adenovirus is a replication-deficient adenoviral vector.

In various embodiments thereof, the tumor volume is decreased by 20% or more. In various embodiments thereof, the tumor size in the subject is decreased by about 25-50%, about 40-70% or about 50-90% or more.

In various embodiments thereof, the therapy reduces the rate of metastasis and/or slows progression of the tumor in the patient.

In various embodiments thereof, the anti-PD-1 antibody is administered within 1 hour after vector-CCL21-DC therapy on days 0, 21 and 42.

In various embodiments thereof, the combination therapy increases the number of tumor antigen specific CD8 and CD4 cells in the subject.

In various embodiments thereof, the high mutational burden of the cancer is determined by a biopsy of the tumor.

In various embodiments thereof, tumor-associated neoantigens are determined.

In various embodiments thereof, the efficacy of combination therapy is followed by elucidation of the neoantigen landscape of the cancer.

In various embodiments thereof, the cancer comprises a mutation selected from KRAS, TP53 (KP) or STK11/LKB1, or any combination thereof.

In various embodiments thereof, the cancer has intratumoral heterogeneity.

In various embodiments thereof, the mutational burden of the cancer is determined by diagnostic assay selected from FoundationOne CDx™, FoundationOne®, FoundationAct®, and FoundationOne®Heme.

In various embodiments thereof, the cancer does not have an activating mutation in the epidermal growth factor receptor or an anaplastic lymphoma kinase gene (ALK) fusion.

In various embodiments thereof, the somatic mutational load and tumor-associated neoantigens before, during and after treatment are used to determine, initiate, prescribe or monitor therapy.

### BRIEF DESCRIPTION OF THE DRAWINGS

FIGS. 1A-1D show the effects of combination therapy in in vivo models of NSCLC.

FIG. 2A-2B are schematics showing the dosing and dose escalation in the phase I trial.

FIG. 3 is a chart outlining the protocol in the event of pembrolizumab toxicity.

FIG. 4 shows the amino acid sequences of human CCL21 (SEQ ID NO: 1) and murine CCL21 (SEQ ID NO: 2).

FIG. 5A-5D shows efficacy of combination therapy in tumors with low mutational burden (FIG. 5A, FIG. 5B) and high mutational burden (FIG. 5C, FIG. 5D).

### DEFINITIONS

As used herein “Programmed cell death protein 1” or “PD-1” refers to a cell surface receptor involved in immune checkpoint blockade mediated by binding to two ligands, PD-L1 and PD-L2. PD-1 binding to its ligands has been shown to reduce T-cell proliferation, cytokine production, and cytotoxic activity.

Polypeptides useful in the methods of the invention encompass both naturally occurring proteins as well as variations and modified forms thereof. By “SLC polypeptide or protein” is meant Secondary Lymphoid-Tissue Chemokine (SLC). Secondary lymphoid tissue cytokine (SLC) polypeptide is also called CCL21. SLC includes naturally occurring mammalian SLCs, and variants and fragments thereof. Preferably the SLC is of human or mouse origin. Most preferably the SLC is human SLC. Human SLC has been cloned and sequenced (see, e.g. Nagira et al. (1997) J Biol Chem 272:19518; the contents of which are incorporated by reference).

As used herein, “CCL21”, “CCL21 gene” or “CCL21 polypeptide or protein” is refers to naturally occurring mammalian CCL21, and variants and fragments thereof. Preferably the CCL21 is human. Consequently the cDNA and amino acid sequences of human CCL21 are known in the art (see, e.g. Accession Nos. BAA21817 and AB002409). The CCL21 agents of the invention comprise native CCL21 polypeptides, native CCL21 nucleic acid sequences, polypeptide and nucleic acid variants, and other components that are capable of blocking the immune response through manipulation of CCL21 expression, activity and receptor binding.

Mouse SLC has also been cloned and sequenced (see, e.g. Accession Nos. NP_035465 and NM_011335). Hromas el al. (1997) J. Immunol 1.59:2554; Hedrick et al. (1997) J. Immunol 159:1589; and Tanabe el al. (1997) J. Immunol 1.59:5671; the contents of which are incorporated herein by reference.

SLC polypeptides for use in the methods disclosed herein can be SLC variants, SLC fragments, analogues, and derivatives. The term “variant” refers to a molecule that exhibits a variation from a described type or norm, such as a protein that has one or more different amino acid residues in the corresponding position(s) of a specifically described protein. An analog is an example of a variant protein. As used herein, the SLC-related gene and SLC-related protein includes the SLC genes and proteins specifically described herein, as well as structurally and/or functionally similar variants or analog of the foregoing. SLC peptide analogs generally share at least about 50%, 60%, 70%, 80%, 90% or more amino acid homology (using BLAST criteria). SLC nucleotide analogs preferably share 50%, 60%, 70%, 80%, 90% or more nucleic acid homology (using BLAST criteria). In some embodiments, however, lower homology is preferred so as to select preferred residues in view of species-specific codon preferences and/or optimal peptide epitopes tailored to a particular target population, as is appreciated by those skilled in the art.

By “analogues” is intended analogues of either, such as described in WO2016/073759, incorporated herein by reference in its entirety. Guidance for preparation, administration, and other aspects of the combination therapy may be found in International application PCT/US2015/059297, published as WO2016/073759, in incorporated herein by reference in its entirety.

The human and murine SLC polypeptide sequences are shown below.

Human SLC
(SEQ ID NO: 1)
MAQSLALSLLILVLAFGIPRTQGSDGGAQDCCLKYSQRKIPAKVVRSYR
KQEPSLGCSIPAILFLPRKRSQAELCADPKELWVQQLMQHLDKTPSPQK
PAQGCRKDRGASKTGKKGKGSKGCKRTERSQTPKGP
Murine SLC
(SEQ ID NO: 2)
MAQMMTLSLLSLDLALCIPWTQGSDGGGQDCCLKYSQKKIPYSIVRGYR
KQEPSLGCPIPAILFLPRKHSKPELCANPEEGWVQNLMRRLDQPPAPGK
QSPGCRKNRGTSKSGKKGKGSKGCKRTEQTQPSRG

An “antigen presenting cell” (APC) is a cell that is capable of activating T cells, and includes, but is not limited to, monocytes/macrophages, B cells and dendritic cells (DCs). The term “dendritic cell” or “DC” refers to any member of a diverse population of morphologically similar cell types found in lymphoid or non-lymphoid tissues. These cells are characterized by their distinctive morphology, high levels of surface MHC-class II expression. DCs can be isolated from a number of tissue sources. DCs have a high capacity for sensitizing MHC-restricted T cells and are very effective at presenting antigens to T cells in situ. The antigens may be self-antigens that are expressed during T cell development and tolerance, and foreign antigens that are present during normal immune processes.

The term “therapeutically effective amount” is used herein to indicate the amount of target-specific composition of the disclosure that is effective to ameliorate or lessen symptoms or signs of disease to be treated.

The terms “treat”, “treated”, “treating” and “treatment”, as used with respect to methods herein refer to eliminating, reducing, suppressing or ameliorating, either temporarily or permanently, either partially or completely, a clinical symptom, manifestation or progression of an event, disease or condition. Such treating need not be absolute to be useful.

The terms “cancer”, “cancerous”, or “malignant” refer to or describe the physiological condition in mammals that is typically characterized by unregulated cell growth. Exemplary cancers contemplated herein are described more fully in the Detailed Description. “Mammal” for purposes of treatment or therapy refers to any animal classified as a mammal, including humans, domestic and farm animals, and zoo, sports, or pet animals, such as dogs, horses, cats, cows, etc. Preferably, the mammal is human.

The “treatment of cancer”, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

As used herein, “pharmaceutical composition” refers to a composition suitable for administration to a subject animal, including humans and mammals. A pharmaceutical composition comprises a pharmacologically effective amount of a virus or antigenic composition of the invention and also comprises a pharmaceutically acceptable carrier. A pharmaceutical composition encompasses a composition comprising the active ingredient(s), and the inert ingredient(s) that make up the pharmaceutically acceptable carrier, as well as any product which results, directly or indirectly, from combination, complexation or aggregation of any two or more of the ingredients. Accordingly, the pharmaceutical compositions of the present invention encompass any composition made by admixing a compound or conjugate of the present invention and a pharmaceutically acceptable carrier.

### DETAILED DESCRIPTION

The present disclosure provides a method for treating cancer, in particular metastatic non-small cell lung carcinoma using a combination of autologous cells comprising the CCL21 gene and a checkpoint inhibitor, such as an anti-PD-1 antibody. In one aspect, the patient has a high mutational burden tumor. Such tumors are particularly amenable to treatment with the combination described herein. The effects of treatment with the compositions of the invention can be observed or monitored in a number of ways, for example, the effects can be observed by the evaluation of a change in a cytokine profile, an evaluation the inhibition of tumor growth or tumor killing, e.g. by observing a reduction in tumor size and/or a reduction in the severity of symptoms associated with the tumor and/or tumor growth, an increased survival rate, to name a few non-limiting examples.

In one aspect, the inclusion and exclusion criteria with respect to clinical factors in conducting the study described here are designed to include a large section of this population. As described below, many patients with a diagnosis of metastatic NSCLC are potential candidates for the proposed trial in that approximately 70% of patients have PD-L1 staining in less than 50% of tumor cells (5).

Study Rationale. The majority of NSCLC patients would also be eligible for the proposed study from the perspective of molecular markers. Patients whose tumors reveal activating mutations in the epidermal growth factor receptor (EGFR) or anaplastic lymphoma kinase gene (ALK) fusion are excluded, as those patients have molecularly targeted first line treatment options. In Western populations, approximately 15% of patients harbor genomic abnormalities in one of these two genes (6-11). Although there are slight differences among studies, 23-30% of NSCLC patients have PD-L1 expression in at least half of their cells using the 22C3 assay (5, 12). The remaining 70%, those with PD-L1 staining in less than 50% of their cells, would meet the eligibility criteria for PD-L1 expression allowing enrollment in the proposed study.

The response rate of NSCLC patients with PD-L1 expression in less than half of the tumor cells has generally been 15% or less (12). Thus, this patient population could substantially benefit from approaches that increase T lymphocyte infiltration and upregulation of PD-L1, both of which would increase the likelihood of response to a PD-1 inhibitor. Although other combinations are under evaluation with PD-1/PD-L1 inhibitors, many of these, such as addition of chemotherapy, radiation or CTLA-4 inhibitors, substantially increase the toxicity beyond what is seen with PD-1 inhibitors alone.

The most commonly proposed reason for the lack of efficacy of PD-1/PD-L1 inhibitors is the absence of T lymphocyte infiltration into the tumor. This leads to the rationale to develop therapies that could recruit T cells to the site of the tumor. In a prior clinical trial of intratumoral Ad-CCL21-DC, T lymphocytes were recruited to the tumor site in more than half of the patients evaluated (22). In accord with these findings, PD-L1 expression increased at the site of the tumor, potentially limiting the efficacy of the Ad-CCL21-DC by preventing the recruited T lymphocytes from mediating anti-tumor responses. Combined Ad-CCL21-DC and PD-1 inhibition has the potential to address these substantial limiting factors for both of the therapies.

The toxicity profile of Ad-CCL21-DC was quite favorable in the inventor's prior study (22). It is hypothesized that the local injection of Ad-CCL21-DC will lead to a systemic T lymphocyte response that will preferentially expand T lymphocyte populations specific for tumor antigens, while approaches such as CTLA-4 inhibitors would induce more global T cell activation. Therefore, it is reasonable to predict herein that the addition of Ad-CCL21-DC will add little toxicity to pembrolizumab while increasing the efficacy.

Although Ad-CCL21-DC appears to be a very different therapy from those already approved for NSCLC, with advances in the field, it is clear that if the combination of Ad-CCL21-DC and pembrolizumab is beneficial, potential impediments to adoption could be overcome. For example, CAR-T cells as cellular therapies are now approved for the treatment of certain hematologic malignancies. Similarly, T-VEC is an approved injectable therapy for the treatment of malignant melanoma. Although the proposed therapy would be an injected cellular therapy, all of the requisite infrastructure required for construction and delivery of such a product are available and are likely to be an even more commonplace at a point when the therapy would potentially be approved.

The proposed study is a phase I study. Pembrolizumab was initially approved based on a phase I trial; however, that trial was particularly large (12). The results of the proposed trial would be expected to show the feasibility of administering Ad-CCL21-DC in combination with pembrolizumab as well as to generate the correlative data that would facilitate further, larger studies. Should this approach be feasible, and potentially efficacious, the most likely strategy would be to then conduct a randomized study comparing pembrolizumab plus Ad-CCL21-DC to platinum-based chemotherapy as the initial systemic therapy for metastatic NSCLC patients with staining of PD-L1 in less than half of cancer cells. If chemotherapy plus a PD-1/PD-L1 inhibitor or any other combination is the approved and preferred option in these patients at the time of a comparative trial, the approved and preferred combination would serve as the control arm.

It is possible that during the conduct of the proposed study, a population of patients based on biomarkers would emerge who would be more likely to benefit from Ad-CCL21-DC plus pembrolizumab. If that is the case and the data is sufficiently strong regarding the subpopulation, it is possible that a confirmatory study would be limited to those patients considered most likely to respond to this innovative approach.

PD-1. PD-1 is an immunoglobulin in the CD28 family. PD-1 is a type I transmembrane glycoprotein containing an extracellular Ig variable-type (V-type) domain involved in ligand binding and a cytoplasmic tail involved in intracellular signaling (48). Binding of PD-1 with PD-L1 (or the other ligand PD-L2) induces the recruitment of SHP-1 and SHP-2 to PD-1, resulting in de-phosphorylation of CD3ζ, PKCθ and ZAP70 essential for T cell receptor (TCR) signaling, and down-regulation of T lymphocyte activation (49, 50). Under healthy conditions, PD-L1 attenuates unwanted immune responses, such as autoimmunity (51).

Pembrolizumab is a humanized anti-PD-1 antibody used in cancer immunotherapy. Pembrolizumab is a highly selective humanized mAb designed to block the interaction between PD-1 and its ligands, programmed cell death ligand 1 (PD-L1) and programmed cell death ligand 2 (PD-L2). Pembrolizumab is an IgG4/kappa isotype with a stabilizing sequence alteration in the Fc region. The theoretical molecular weights of the heavy and light chains derived from the amino acid sequences, excluding glycosylation, are 49.4 kiloDaltons (KDa) and 23.7 KDa, respectively. The detailed information on pembrolizumab is described in the Investigator's Brochure and approved labeling.

Clinical trials testing the safety and efficacy of pembrolizumab in treating NSCLC patients have led to the approval of the agent for this disease. The KEYNOTE-001 study revealed responses in approximately 20% of the patients with a modest side-effect profile (12). Importantly, patients with >50% PD-L1 baseline tumor staining experienced greater benefit from anti-PD-1 therapy than those with <50% tumor PD-L1 expression, with the ORR defined by Response Evaluation Criteria in Solid Tumors (RECIST) criteria of 45.2% in patients with >50% PD-L1 staining, versus 16.5% in patients with 1-49% PD-L1 staining and 10.7% in patients with <1% PD-L1 staining. In the KEYNOTE-010 phase II/III study, 2 mg/kg and 10 mg/kg q3w of pembrolizumab showed significant benefit over 75 mg/m2 q3w docetaxel in randomized, stage IV pre-treated patients with >1% PD-L1 staining (17). In the KEYNOTE-024 study, 200 mg of pembrolizumab showed significant benefit over investigators' choice of standard of care chemotherapy in treatment-naïve patients with >50% PD-L1 staining (5). Despite robust and durable responses to anti-PD-1 therapy in a subgroup of NSCLC patients, most patients do not respond to PD-1 checkpoint inhibitors as single agents (12). Therefore, rational and effective combination strategies with PD-1 inhibitors are needed to enhance the efficacy of the anti-PD1 therapy in advanced NSCLC patients.

Antibodies to PD-1 have been described in U.S. Pat. Nos. 8,735,553; 8,617,546; 8,008,449; 8,741,295; 8,552,154; 8,354,509; 8,779,105; 7,563,869; 8,287,856; 8,927,697; 8,088,905; 7,595,048; 8,168,179; 6,808,710; 7,943,743; 8,246,955; and 8,217,149.

It is contemplated that any known anti-PD-1 antibody can be used in the present methods. In various embodiments, the anti-PD-1 antibody inhibits or blocks binding of the PD-1 receptor to one or both of its ligands, PD-L1 and PD-L2. In exemplary aspects, the monoclonal antibody that specifically binds to PD-1 is Nivolumab (BMS936558; Bristol Meyers Squibb), Pembrolizumab (MK-3475; Merck), Pidilizumab (CT-011; CureTech), Lambrolizumab, BMS-936559, Atezolizumab, or AMP-224 (GSK/Amplimmune), AMP224 (MedImmune); AUNP12 (Dr. Reddy's Laboratories Ltd.); BGB108 (BeiGene); MCLA134 (Merus BV); MEDIO680 (MedImmune); PDR001 (Novartis); REGN2810 (Regeneron/Sanofi); SHR1210 (Jiangsu Hengrui Medicine/Incyte); STIA110X (Sorrento); STIA1110 (Sorrento); TSR042 (AnaptysBio/Tesaro). In exemplary aspects, the monoclonal antibody that specifically binds to PD1-L1 is BMS-936559 (BMS/Ono), MPDL3280A (Roche/Genentech), or MEDI-4736 (MedImmune), MSB0010718C (Merck/Serono), ALN-PDL (Alnylam); BGBA317 (BeiGene); KD033 (Kadmon Corp.); KY1003 (Kymab Ltd.); STIA100X (Sorrento); STIA1010 (Sorrento); STIA1011 (Sorrento); STIA1012 (Sorrento); and STIA1014 (Sorrento).

Inhibitors of PD-L1 have also been shown to be effective at inhibiting solid tumors in bladder cancer, head and neck cancer, and gastrointestinal cancers (Herbst R S et al., J Clin Oncol., 31: 3000 (2013); Heery C R et al., J Clin Oncol., 32: 5s, 3064 (2014); Powles T et al., J Clin Oncol, 32: 5s, 5011(2014); Segal N H et al., J Clin Oncol., 32: 5s, 3002 (2014)).

CCL21 Modified Dendritic Cells. CCL21, also known as Secondary lymphoid tissue chemokine (SLC), Exodus 2 or 6Ckine, is a high endothelial-derived CC chemokine normally expressed in high endothelial venules and in T-cell zones of spleen and lymph node, that strongly attracts naive T cells and Dendritic cells (DCs) (Cyster et al., J. Exp. Med., 189: 447-450, 1999.24; Ogata et al., Blood, 93: 3225-3232, 1999; Chan et al., Blood, 93: 3610-3616, 1999; Hedrick et al., J. Immunol., 159: 1589-1593, 1997; Hromas et al., J. Immunol., 159: 2554-2558, 1997; Nagira et al., J. Biol. Chem., 272: 19518-19524,1997; Tanabe et al., J. Immunol., 159: 5671-5679, 1997; Willimann et al., Eur. J. Immunol., 28: 2025-2034, 1998). CCL21 mediates its effects through two specific G protein-coupled seven-transmembrane domain chemokine receptors, CCR7 and CXCR3 (Yoshida et al., J. Biol. Chem. 273:7118; Jenh et al., J. Immunol. 162:3765). Whereas CCR7 is expressed on naive T cells and mature DC, CXCR3 is expressed preferentially on Th1 cytokine-producing lymphocytes with memory phenotype (Yoshida et al., J. Biol. Chem. 273:7118; Jenh et al., J. Immunol. 162:3765).

The capacity of CCL21 to chemoattract DCs (Kellermann et al., J. Immunol., 162: 3859-3864, 1999) is a property shared with other chemokines (Sallusto et al., Eur. J. Immunol., 28: 2760-2769, 1998; Sozzani et al., J. Immunol., 161: 1083-1086, 1998; Dieu et al., J. Exp. Med., 188: 373-386, 1998). However, SLC may be distinctly advantageous because of its capacity to elicit a Type 1 cytokine response invivo (Sharma et al., J. Immunol., 164: 4558-4563, 2000). CCL21 recruits both naive lymphocytes and antigen stimulated DCs into T-cell zones of secondary lymphoid organs, colocalizing these early immune response constituents and culminating in cognate T-cell activation (Cyster et al., J. Exp. Med., 189: 447-450, 1999.24).

Dendritic cells are bone marrow-derived professional APCs that process and present antigens to facilitate activation and expansion of antigen-specific T lymphocytes (52, 53). Recent studies demonstrate that tumor-residing BATF3-driven CD103+ (mouse)/CD141+ (human) DCs are required for tumor antigen trafficking, effector T lymphocyte infiltration and T lymphocyte antitumor immunity (54, 55). In addition, CXCL10 produced by CD103+DCs is required for effector T lymphocyte migration (55). A variety of strategies has been utilized to exploit activated DC in cancer immunotherapy (56-59). Chemokines are a group of homologous, yet functionally divergent proteins that directly mediate leukocyte migration and activation, as well as angiogenesis (60). CCL21 is a chemokine that strongly attracts T lymphocytes and DC to T lymphocyte zones by interacting with chemokine receptors CXCR3 and CCR7 (40, 41), co-localizing these early immune response constituents to promote T lymphocyte activation (41). In addition, CCL21 is a potent angiostatic agent (61), thus adding further support for its use in cancer therapy.

Intratumoral (IT) administration of the recombinant CCL21 mediated T lymphocyte-dependent antitumor responses in 2 independent syngeneic murine lung cancer models, L1C2 and 3LLm, was studied (62). Consistently, IT injection of CCL21 significantly increased the infiltration of CD4+ and CD8+T lymphocytes and DC into both the tumor and draining lymph nodes. Accompanying these cell infiltrates were increases in IFN□, CXCL9, CXCL10, GM-CSF and IL-12, with a concomitant decrease in the immunosuppressive molecules PGE-2 and TGFβ. The efficacy of CCL21-mediated antitumor responses required induction of IFN□, CXCL9 and CXCL10 (63). In addition, systemic antitumor immune responses were demonstrated in CCL21-treated tumor-bearing mice (62). Similarly, the antitumor activity of CCL21 was demonstrated in a transgenic mouse model of spontaneous murine bronchoalveolar cell carcinoma (64).

Cancer immunotherapy employing PD-1/PD-L1 checkpoint blockade can induce robust and durable responses in a subgroup of patients with metastatic cancers, including melanoma and NSCLC; however, the majority of the patients do not respond to PD-1 inhibitors as single agents due to primary, adaptive or acquired resistance to cancer immunotherapy (79). Strategies to enhance the effectiveness of checkpoint blockade will benefit a larger population of cancer patients, including those with advanced NSCLC.

Vectors. A method for delivery of a CCL21 expression construct involves the use of an expression vector. Exemplary vectors include viral vectors, liposomes or plasmid vector, as well as other gene delivery vectors. Viral vectors include adenovirus, an adeno-associated virus, a lentivirus, a retrovirus, a vaccinia virus, modified Ankara virus, and Vesicular stomatitis virus.

Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct in host cells with complementary packaging functions and (b) to ultimately express a heterologous gene of interest that has been cloned therein. See e.g. International Patent application PCT/US15/59297, incorporated herein by reference.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences (Grunhaus and Horwitz, 1992). In contrast to retrovirus, the adenoviral infection of host cells does not result in chromosomal integration because wild-type adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The El region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNAs for translation.

In various embodiments, the vector is a replication deficient adenoviral vector.

In various embodiments, the adeno-associated virus (AAV) is selected from the group consisting of AAV1, AAV2, AAV3, AAV4, AAV5, AAV6, AAV7, AAV8 or combinations thereof.

Methods of Use. Exemplary conditions or disorders that can be treated with the proposed combination therapy include cancers, such as esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIIA skin melanoma; stage IIIB skin melanoma, stage IIIC skin melanoma; stage IV skin melanoma, malignant melanoma of head and neck, lung cancer, non-small cell lung cancer (NSCLC), squamous cell non-small cell lung cancer, breast cancer, recurrent metastatic breast cancer, hepatocellular carcinoma, Hodgkin's lymphoma, follicular lymphoma, non-Hodgkin's lymphoma, advanced B-cell NHL, HL including diffuse large B-cell lymphoma (DLBCL), multiple myeloma, chronic myeloid leukemia, adult acute myeloid leukemia in remission; adult acute myeloid leukemia with Inv(16)(p13.1q22); CBFB-MYH11; adult acute myeloid leukemia with t(16;16)(p13.1;q22); CBFB-MYH11; adult acute myeloid leukemia with t(8;21)(q22;q22); RUNX1-RUNX1T1; adult acute myeloid leukemia with t(9;11)(p22;q23); MLLT3-MLL; adult acute promyelocytic leukemia with t(15;17)(q22;q12); PML-RARA; alkylating agent-related acute myeloid leukemia, chronic lymphocytic leukemia, Richter's syndrome; Waldenstrom's macroglobulinemia, adult glioblastoma; adult gliosarcoma, recurrent glioblastoma, recurrent childhood rhabdomyosarcoma, recurrent Ewing sarcoma/peripheral primitive neuroectodermal tumor, recurrent neuroblastoma; recurrent osteosarcoma, colorectal cancer, MSI positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; recurrent cervical carcinoma; stage IVA cervical cancer; stage IVB cervical cancer, anal canal squamous cell carcinoma; metastatic anal canal carcinoma; recurrent anal canal carcinoma, recurrent head and neck cancer; carcinoma, squamous cell of head and neck, head and neck squamous cell carcinoma (HNSCC), ovarian carcinoma, colon cancer, gastric cancer, advanced GI cancer, gastric adenocarcinoma; gastroesophageal junction adenocarcinoma, bone neoplasms, soft tissue sarcoma; bone sarcoma, thymic carcinoma, urothelial carcinoma, recurrent merkel cell carcinoma; stage III merkel cell carcinoma; stage IV merkel cell carcinoma, myelodysplastic syndrome and recurrent mycosis fungoides and Sezary syndrome.

In some embodiments, in the cancer treated with the present method less than 50% of tumor cells express the PD-L1 protein on their surface. In various embodiments, the cancers have greater than 50% PD-L1 staining on their cell surface, and therefore greater than 50% of tumor cells express the PD-L1 protein on their surface.

It is further contemplated that the present methods are useful in subjects treated with first line pembrolizumab plus chemotherapy, or, alternatively, in subjects that fail initial therapy with this combination.

In some embodiments, cancers that can be treated with the present methods include metastatic NSCLC and other solid tumors as described herein.

It is contemplated that the methods herein reduce tumor size or tumor burden in the subject, and/or reduce metastasis in the subject. In various embodiments, tumor size or tumor volume in the subject is decreased by about 25-50%, about 40-70% or about 50-90% or more. In various embodiments, the methods reduce the tumor size or tumor volume by 10%, 20%, 30%, or more. In various embodiments, the methods reduce tumor size or tumor volume by 10%, 15%, 20%, 25%, 30%, 35%, 40%, 45%, 50%, 55%, 60%, 65%, 70%, 75%, 80%, 85%, 90%, 95% or 100%.

It is contemplated that the methods herein reduce tumor burden, and also reduce or prevent the recurrence of tumors once the cancer has gone into remission.

It is also contemplated that administration of the vector-CCL21-DC increases CD8 T cell infiltration into a tumor. In various embodiments, the CD8 cells are increased by 2-fold or more in the treated subject compared to a subject not receiving combination therapy. It is provided that the vector-CCL21-DC increases PD-L1 expression in a tumor.

In various embodiments, the dendritic cells are administered intratumorally, intravenously, intra-arterially, intraperitoneally, intranasally, intramuscularly, intradermally or subcutaneously, or via CT-guided or bronchoscopic IT injection. In various embodiments, the checkpoint inhibitor is administered intravenously.

The route of administration of the SLC, dendritic cells or checkpoint inhibitor will vary depending on the desired outcome. Generally for initiation of an immune response, injection of the agent at or near the desired site of inflammation or response is utilized. Alternatively other routes of administration may be warranted depending upon the disease condition. That is, for suppression of neoplastic or tumor growth, injection of the pharmaceutical composition at or near the tumor site is preferred. Alternatively, for prevention of graft rejection, systemic administration may be used. Likewise, for the treatment or prevention of autoimmune diseases systemic administration may be preferred. Examples of routes of systemic administration include parenteral, e.g., intravenous, intradermal, subcutaneous, oral (e.g., inhalation) transdermal (topical), transmucosal, and rectal administration. Solutions or suspensions used for parenteral, intradermal, or subcutaneous application can include the following components: a sterile diluent such as water for injection, saline solution; fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose.

In one embodiment, the pharmaceutical composition can be delivered via slow release formulation or matrix comprising SLC protein or DNA constructs suitable for expression of SLC protein into or around a site within the body. In this manner, a transient lymph node can be created at a desired implant location to attract dendritic cells and T cells initiating an immune response.

It is contemplated that the anti-PD-1 antibody is administered every three weeks starting on day 0. In various embodiments, the anti-PD-1 antibody is pembrolizumab administered at a dose of 200 mg every three weeks.

In various embodiments, the Ad-CCL21-DC is administered in a dose from 5×106 cells/injection to 3×107 cells/injection, e.g., 5×106, 1×107, or 3×107 cells/injection. It is contemplated that the dendritic cells comprising a vector-CCL21, such as an Adenovirus-CCL21 (Ad-CCL21-DC) construct, are administered at 3-week intervals, e.g., on days 0, 21, and 42.

It is further contemplated that other adjunct therapies may be administered, where appropriate. For example, the patient may also be administered surgical therapy, chemotherapy, a cytotoxic agent, photodynamic therapy or radiation therapy where appropriate.

A wide variety of chemotherapeutic agents may be used in combination with the combination therapy of the present invention. These can be, for example, agents that directly cross-link DNA, agents that intercalate into DNA, and agents that lead to chromosomal and mitotic aberrations by affecting nucleic acid synthesis. A variety of chemotherapeutic agents are intended to be of use in the combined treatment methods disclosed herein. Chemotherapeutic agents contemplated as exemplary include, e.g., etoposide (VP-16), adriamycin, 5-fluorouracil (5FU), camptothecin, actinomycin-D, mitomycin C, cisplatin (CDDP) and even hydrogen peroxide. In other embodiments, any surgical intervention may be practiced in combination with the present invention. In connection with radiotherapy, any mechanism for inducing DNA damage locally within tumor cells is contemplated, such as y-irradiation, X-rays, UV-irradiation, microwaves and even electronic emissions and the like. The directed delivery of radioisotopes to tumor cells is also contemplated, and this may be used in connection with a targeting antibody or other targeting means. Cytokine therapy also has proven to be an effective partner for combined therapeutic regimens. Various cytokines may be employed in such combined approaches. Examples of cytokines include IL-1, IL-Iβ, IL-2, IL-3, IL-4, IL-5, IL-6, IL-7, IL-8, IL-9, IL-10, IL-11, IL-12, IL-13, TGF-β, GM-CSF, M-CSF, TNFa, TNPβ, LAF, TCGF, BCGF, TRF, BAF, BDG, MP, LIF, OSM, TMF, PDGF, IFN-a, IFN-β, IFN-γ. Cytokines are administered according to standard regimens, consistent with clinical indications such as the condition of the patient and relative toxicity of the cytokine. Below is an exemplary, but in no way limiting, table of cytokine genes contemplated for use in certain embodiments of the present invention.

As will be understood by those of ordinary skill in the art, the appropriate doses of chemotherapeutic agents will be generally around those already employed in clinical therapies wherein the chemotherapeutics are administered alone or in combination with other chemotherapeutics. By way of example only, agents such as cisplatin, and other DNA alkylating may be used. Cisplatin has been widely used to treat cancer, with efficacious doses used in clinical applications of 20 mg/in2 for 5 days every three weeks for a total of three courses. Cisplatin is not absorbed orally and must therefore be delivered via injection intravenously, subcutaneously, intratumorally or intraperitoneally.

Agents that directly cross-link nucleic acids, specifically DNA, are envisaged and are shown herein, to eventuate DNA damage leading to a synergistic antineoplastic combination. Agents such as cisplatin, and other DNA alkylating agents may be used.

Further useful agents include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/in2 at 21-day intervals for adriamycin, to 35-50 mg/in2 for etoposide intravenously or double the intravenous dose orally.

Agents that disrupt the synthesis and fidelity of polynucleotide precursors may also be used. Particularly useful are agents that have undergone extensive testing and are readily available. As such, agents such as 5-fluorouracil (5-FU) are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Plant alkaloids such as taxol are also contemplated for use in certain aspects of the present invention. Taxol is an experimental antimitotic agent, isolated from the bark of the ash tree, Taxus brevifolia. It binds to tubulin (at a site distinct from that used by the vinca alkaloids) and promotes the assembly of microtubules. Taxol is currently being evaluated clinically; it has activity against malignant melanoma and carcinoma of the ovary. Maximal doses are 30 mg/m2 per day for 5 days or 210 to 250 mg/m 2 given once every 3 weeks. Of course, all of these dosages are exemplary, and any dosage in-between these points is also expected to be of use in the invention.

Other exemplary chemotherapeutic agents that are useful in connection with combined therapy are listed in Table B of WO2016/073759, incorporated herein by reference in its entirety. Each of the agents listed therein are exemplary and by no means limiting. The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

As described herein, in one embodiment, any of the methods described herein are amenable to treatment of patients having a tumor with a high mutational burden. In one embodiment, the tumor is a NSCLC, or another solid tumor, such as but not limited to a squamous cell or non-squamous cell tumor. The presence of a high tumor mutational burden in a tumor can be determined from a biopsy using any one of a number of diagnostic assays, e.g., from FoundationOne (Cambridge, Mass.), such as FoundationOne CDx™, FoundationOne®, FoundationAct®, or FoundationOne®Heme.

In non-limiting embodiments, mutations such as KRAS, TP53 (KP) and STK11/LKB1 may be present in the tumor. In other embodiments, high nonsynonymous mutational burden is present. In one embodiment, high mutational load from exposure to carcinogens underly the tumor and its susceptibility to the combination treatment described herein. As noted herein, in one embodiment, the mutational load of a tumor is first assessed, and treatment as described herein initiated for tumors of high mutational load.

As will be noted in the examples below, the efficacy of the combination therapy on tumors with high mutational burden is demonstrated in a model of human tumors with high mutational burden. Recent studies demonstrate that human NSCLC (over 85% of lung cancers) possesses high nonsynonymous mutational burden, a biomarker associated with clinical benefit of PD-1 blockade. KRAS mutations are the most prevalent oncogenic driver in NSCLC (˜25% in LUAC), and recent studies reveal that co-occurring mutations in the STK11/LKB1 (KL) and TP53 (KP) tumor suppressor genes define distinct subgroups of NSCLC with unique TME immune profiles. LKB1 mutation has recently been identified as a major driver of primary resistance to PD-1 blockade in KRAS-mutant lung adenocarcinoma (LUAC). The foregoing are merely non-limiting examples of mutations in tumors amenable to treatment by the methods described herein.

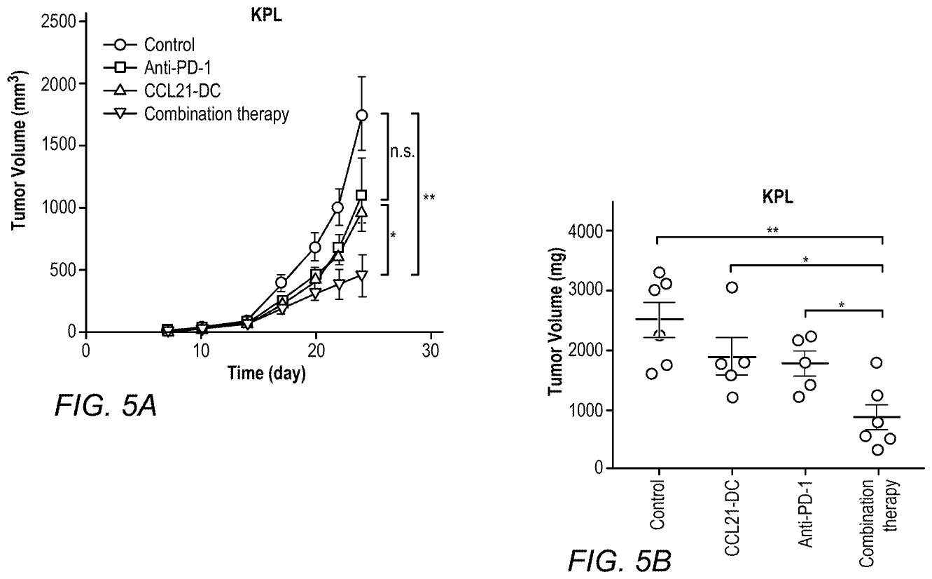

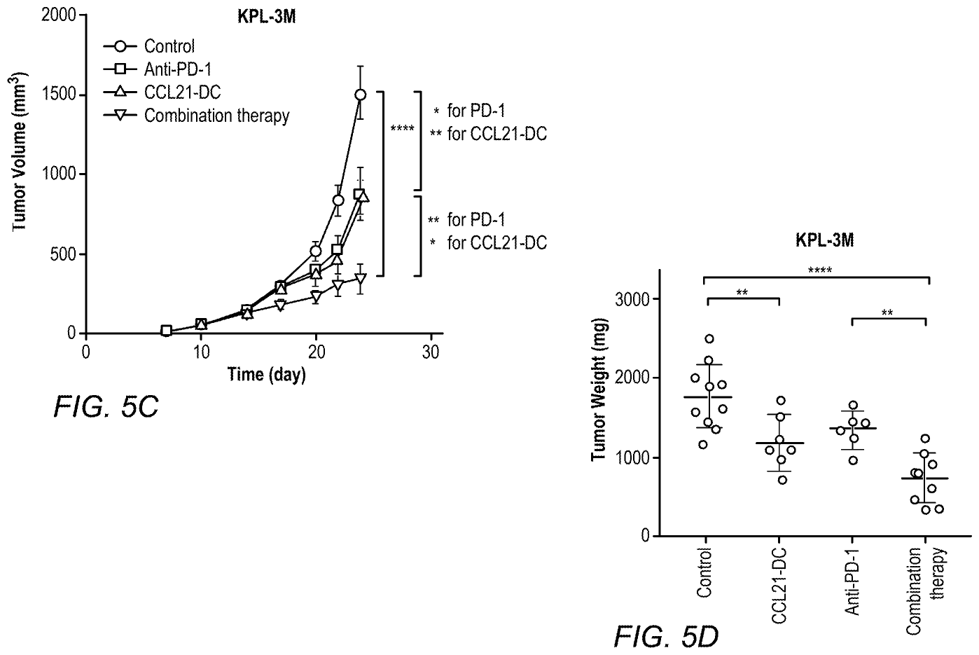

In the example herein, a genetically engineered mouse model of lung cancer, such as KrasG12D (LKR13), KrasG12D;P53−/−(KP) and KrasG12D;P53−/−;Lkb1−/− (KPL), possess common driver mutations of human NSCLC. To increase mutational burden to model human NSCLC which frequently possess high tumor mutational burden (TMB), exposure of LKR13, KP, and KPL cells to tobacco carcinogen methyl-nitrosourea (MNU) recapitulates the mutational landscape of human NSCLC. As described in the example, whole exome sequencing (WES) of these mutant cell lines revealed significant increases in mutational loads and intratumoral heterogeneity. FIGS. 5C and 5D show that the combination therapy described here resulted in effective tumor eradication (*, P<0.05; **, P<0.005; ****, P<0.00005) thus indicating that CCL21-DC improves the efficacy of anti-PD-1 for lung cancer treatment and in particular, efficacy in a high tumor burden model.

This, in one embodiment, all of the foregoing descriptions of clinical trial design, dosing designs and regiments, and other aspects of the treatment of cancer are applicable to patients with tumors with high mutational burden. Moreover, aspects of study design and treatment described in PCT/US15/59297 are applicable to patients with tumors having high mutational burden. Non-limiting aspects at least or about 1×10⁵ or at least or about 1×10⁶ cells comprising and expressing the polynucleotide encoding the SLC polypeptide are administered to the subject. In exemplary aspects, at least or about 2×10⁶ cells, at least or about 3×10⁶ cells, at least or about 4×10⁶ cells, at least or about 5×10⁶ cells, at least or about 6×10⁶ cells, at least or about 7×10⁶ cells, at least or about 8×10⁶ cells, at least or about 9×10⁶ cells, at least or about 1×10⁷ cells, at least or about 2×10⁷ cells, or at least or about 3×10⁷ cells comprising and expressing the polynucleotide encoding the SLC polypeptide are administered to the subject. In exemplary aspects, the cells produce a sufficient amount of SLC in a given time period. In exemplary aspects, the cells produce at least or about 0.10 ng of SLC per 1×10⁶ cells in a 24-hour period. In exemplary aspects, the cells produce at least or about 0.15 ng of SLC per 1×10⁶ cells in a 24-hour period. In exemplary aspects, the cells produce at least or about 0.20 ng of SLC per 1×10⁶ cells in a 24-hour period. In exemplary aspects, the cells produce at least or about 0.25 ng of SLC per 1×10⁶ cells in a 24-hour period. In exemplary aspects, the cells produce at least or about 0.30 ng of SLC per 1×10⁶ cells in a 24-hour period. In exemplary aspects, the cells produce at least or about 0.35 ng of SLC per 1×10⁶ cells in a 24-hour period. In exemplary aspects, the cells produce at least or about 0.40 ng of SLC per 1×10⁶ cells in a 24-hour period. In exemplary aspects, the cells produce at least or about 0.45 ng of SLC per 1×10⁶ cells in a 24-hour period. In exemplary aspects, the cells produce at least or about 0.50 ng of SLC per 1×10⁶ cells in a 24-hour period. In exemplary aspects, 1 to 30 million cells produce about 0.2 to 0.45 ng (e.g., 0.292 ng to about 0.413 ng) of SLC per 1×10⁶ cells in a 24-hour period.

In exemplary aspects, the SLC polypeptides, SLC variants, SLC fragments, SLC analogues, SLC derivatives, SLC polynucleotides encoding said polypeptides, variants, or fragments, and/or the SLC agents described herein is/are administered before administration of the immune checkpoint inhibitor. In exemplary aspects, the SLC polypeptides, SLC variants, SLC fragments, SLC analogues, SLC derivatives, SLC polynucleotides encoding said polypeptides, variants, or fragments, and/or the SLC agents described herein is/are administered about 1 day, about 2 days, about 3 days, about 4 days, about 5 days, about 6 days, about 1 week, about 2 weeks, about 3 weeks, or about 4 weeks before administration of the immune checkpoint inhibitor.

In exemplary aspects, the SLC polypeptides, SLC variants, SLC fragments, SLC analogues, SLC derivatives, SLC polynucleotides encoding said polypeptides, variants, or fragments, and/or the SLC agents described herein is/are administered after administration of the immune checkpoint inhibitor. In exemplary aspects, the SLC polypeptides, SLC variants, SLC fragments, SLC analogues, SLC derivatives, SLC polynucleotides encoding said polypeptides, variants, or fragments, and/or the SLC agents described herein is/are administered about 1 day, about 2 days, about 3 days, about 4 days, about 5 days, about 6 days, about 1 week, about 2 weeks, about 3 weeks, or about 4 weeks after administration of the immune checkpoint inhibitor.

In exemplary aspects, the SLC polypeptides, SLC variants, SLC fragments, SLC analogues, SLC derivatives, SLC polynucleotides encoding said polypeptides, variants, or fragments, and/or the SLC agents described herein is/are administered concurrently with the immune checkpoint inhibitor. In various embodiments, the agents are administered in a separate formulation and administered concurrently, with concurrently referring to agents given within 30 minutes of each other. The methods also provide that the SLC composition and checkpoint inhibitor are administered with a second agent, e.g., a chemotherapeutic, which can be administered prior to administration with either the SLC composition and/or the checkpoint inhibitor, after administration with either the SLC composition and/or the checkpoint inhibitor, or administered concurrent with either the SLC composition and/or checkpoint inhibitor.

In various embodiments, it is contemplated the SLC agent and checkpoint inhibitor may be given simultaneously, in the same formulation.

In exemplary aspects, the SLC polypeptides, SLC variants, SLC fragments, SLC analogues, SLC derivatives, SLC polynucleotides encoding said polypeptides, variants, or fragments, and/or the SLC agents described herein is/are administered before and after administration of the immune checkpoint inhibitor administration. In exemplary aspects, the SLC polypeptides, SLC variants, SLC fragments, SLC analogues, SLC derivatives, SLC polynucleotides encoding said polypeptides, variants, or fragments, and/or the SLC agents described herein is/are administered before administration of the immune checkpoint inhibitor, after administration of the immune checkpoint inhibitor, and concurrently with the immune checkpoint inhibitor.

In exemplary aspects, the (i) SLC polypeptide, (ii) polynucleotide encoding the SLC polypeptide, (iii) cell comprising the polynucleotide, or (iv) combination thereof, is administered to the subject more than once. In exemplary aspects, the (i) SLC polypeptide, (ii) polynucleotide encoding the SLC polypeptide, (iii) cell comprising the polynucleotide, or (iv) combination thereof, is administered to the subject twice weekly, once weekly, once every 2 weeks, once every 3 weeks, or once monthly. In exemplary aspects, the immune checkpoint inhibitor is administered to the subject more than once. In exemplary aspects, the immune checkpoint inhibitor is administered to the subject twice weekly, once weekly, once every 2 weeks, once every 3 weeks, or once monthly.

In exemplary aspects, the subject comprises a solid tumor and the cells are administered to the subject intratumorally. In alternative aspects, the cells are administered to the subject parenterally, e.g., intravenously or subcutaneously.

In exemplary aspects, the method comprises intravenously administering to the subject an immune checkpoint inhibitor about once every two weeks at a dosage within about 1 to about 20 mg/kg and intratumorally administering to the subject about 1 to about 30 million cells comprising and expressing an SLC polynucleotide encoding an SLC polypeptide comprising the amino acid sequence of SEQ ID NO: 1 or SEQ ID NO: 2. In exemplary aspects, the method comprises intravenously administering to the subject an immune checkpoint inhibitor about once every two weeks at a dosage within about 1 to about 20 mg/kg and intratumorally administering to the subject an SLC polynucleotide encoding an SLC polypeptide comprising the amino acid sequence of SEQ ID NO: 1 or SEQ ID NO: 2.

In exemplary aspects, the cells or SLC polypeptide are administered to the subject about 2 weeks prior to the first administration of the immune checkpoint inhibitor. In exemplary aspects, the cells or SLC polypeptide are administered to the subject monthly after the first administration of cells or SLC polypeptide. In exemplary aspects, the immune checkpoint inhibitor is administered to the subject every 2 weeks starting two weeks after the first administration of the immune checkpoint inhibitor.

### EXAMPLES

### Example 1. CCL21 and PD-1 Combination Therapy is Effective in Models of NSCLC

In vivo models of NSCLC (3LL model and KRAS-mutant LKR13 model) were used to measure the efficacy of a combination of CCL21 and anti-PD-1 antibody on tumor growth and immune stimulation. Initial results were reported in Co-owned International Patent Application PCT/US15/59297.

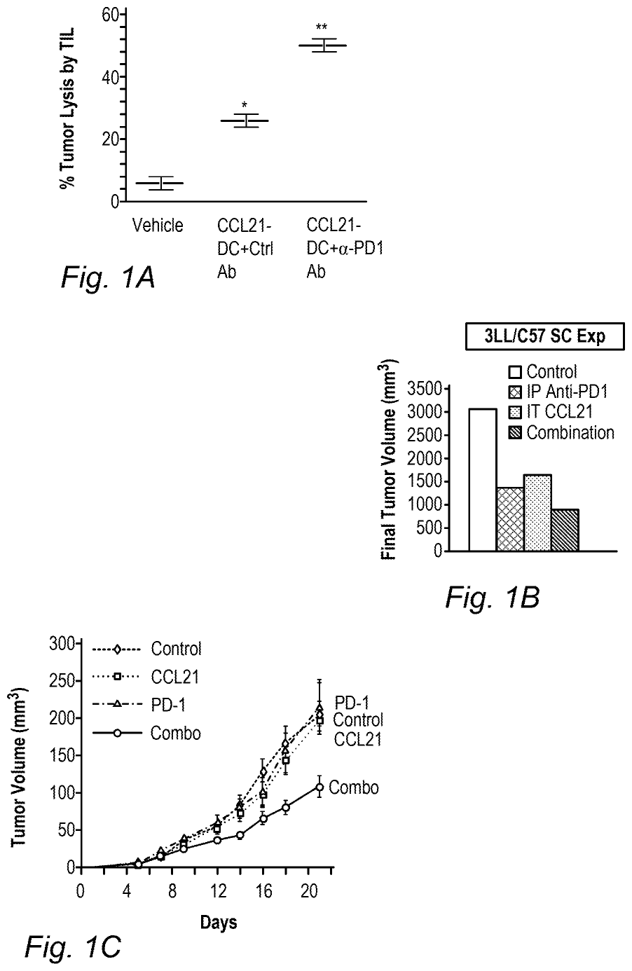

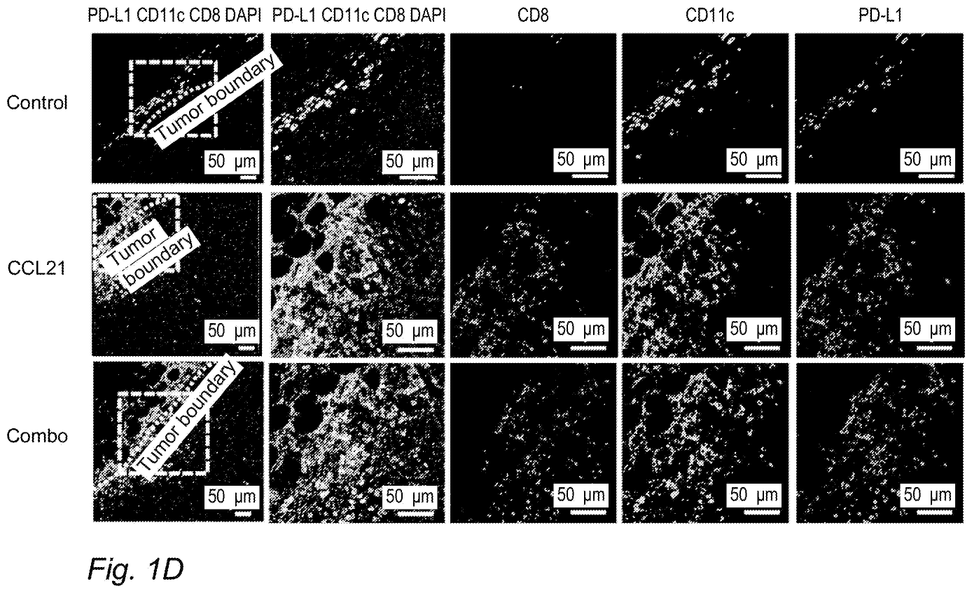

Results show that combination therapy outperforms both mono-therapies in murine models of NSCLC. FIG. 1A shows in vitro activity of TILs from Ad-CCL21-DC-treated mice bearing 3LL tumors was enhanced by PD-1 blockade. In vitro T cell cytolytic activity against autologous tumor was evaluated in the presence of anti-PD-1 (1 μg/ml) or control antibody (1 μg/ml). Values reflect mean±SEM, n=8 mice/group, *P<0.05 relative to Vehicle, **P<0.05 relative to Control antibody.

FIG. 1B shows the effects of treatment in an in vivo 3LL murine lung cancer model. Mice bearing 50 mm3 SC 3LL tumors were treated with i) vehicle control, ii) CCL21 (1.5 g/dose IT), iii) Anti-PD-1 (200 μg/dose IP), or iv) combination therapy every 5 days for 3 times. Results show combination therapy had lower tumor volume compared to single agent or vehicle treatment. A KRAS-mutant LKR13 model was also used. On d5 post-tumor inoculation (1.5×106 LKR13 delivered SC), 129/E mice bearing <25 mm3 tumors were treated as in FIG. 1B every other day throughout the study. LKR13 tumors were resistant to mono-therapies, while the combination reduced tumor growth (FIG. 1C) out as far as 20 days post induction.

Multiplex immunofluorescence (MIF) analysis of the tumors from FIG. 1C showed increases in the tumor PD-L1 expression and the influx of CD8+ T cells at the tumor margin and within the tumor in CCL21-treated and the combination groups as compared to control (FIG. 1D).

Consistently, the combination of IT CCL21 and IP anti-PD-1 outperformed both mono-therapies to elicit antitumor immunity in 2 independent syngeneic murine lung cancer models (FIGS. 1B and 1C). Increased tumor CD8+ T cell infiltration and PD-L1 expression were also observed in response to CCL21 treatment in the KLR13 tumor model (FIG. 1D), consistent with previous data.

### Example 2. Phase I Trial of CCL21 and Anti-PD-1 Antibody to Treat Non-Small Cell Lung Cancer

Although IT administration of Ad-CCL21-DC induced CD8+ T lymphocyte tumor infiltration and systemic antitumor responses in a subset of advanced NSCLC patients, we observed increased PD-L1 expression in the TME of Ad-CCL21-DC-treated patients, suggesting that tumor-mediated impairment of T lymphocyte function may be forestalling a more robust CCL21-mediated antitumor response. Similarly, the lack of efficacy for PD-1/PD-L1 inhibitors could potentially be combated by enhanced T lymphocyte infiltration and augmented antigen presenting cell function. Therefore, we propose to combine Ad-CCL21-DC therapy with PD-1 inhibition to amplify host antitumor immunity in NSCLC patients. This may be particularly important in patients with low or absent baseline tumor PD-L1 expression, who may also demonstrate minimal tumor T lymphocyte infiltration and most often do not respond to PD-1 inhibition alone. The current goal is to determine the potential of Ad-CCL21-DC to stimulate specific antitumor immune responses that enhance anti-PD-1 efficacy in NSCLC patients.

The Example below describes a Phase I trial to determine the safety and maximum tolerated dose (MTD) of intratumoral (IT) injection of CCL21 gene-modified DC (Ad-CCL21-DC) when combined with intravenous pembrolizumab in patients with previously untreated, advanced NSCLC, whose tumors express PD-L1 in less than 50% of tumor cells.

Another aspect of the study will evaluate the objective response rate (ORR) in subjects treated with the dose established during dose escalation (ExD) of IT injection of Ad-CCL21-DC when administered with intravenous pembrolizumab in patients with previously untreated, advanced NSCLC whose tumors express PD-L1 in less than 50% of tumor cells. Currently, the PD-L1 immunostaining is a standard procedure (22C3 assay) routinely performed at the diagnosis of the disease. Approximately 70% of NSCLC patients have PD-L1 expression in <50% of the tumor cells using the 22C3 assay (5, 12).

Secondary objectives of the Ph. I study include to define the adverse event (AE) profile of IT injection of Ad-CCL21-DC (determined during dose escalation) when administered with intravenous pembrolizumab in patients with previously untreated, advanced NSCLC whose tumors express PD-L1 in less than 50% of tumor cells, and to determine relationship of AEs to study treatment. Also contemplated is determining drug target activity by analyzing serial pre- and post-treatment biopsies and blood specimens of IT injection of Ad-CCL21-DC when administered with intravenous pembrolizumab in patients with previously untreated, advanced NSCLC whose tumors express PD-L1 in less than 50% of tumor cells.

Inclusion Criteria

The following subjects can be included in the study:
1. Adults over the age of 18 capable of giving informed consent.
2. Stage IV pathologically proven NSCLC.
3. Staining for PD-L1 in less than half of the tumor cells using the CC23 antibody (0% staining is acceptable).
4. Measurable disease by RECIST v1.1 Guidelines.
5. ECOG performance status of 0, 1.
6. Must be naïve to systemic treatment for NSCLC. Patients who received adjuvant or neo-adjuvant chemotherapy are eligible if at least 6 months have passed since last treatment.
7. Adequate renal function (defined as BUN≤40 mg/dL or serum creatinine ≤2 mg/dL).
8. Adequate liver function (defined as serum total bilirubin ≤2× the upper limits of normal (ULN), or serum transaminases ≤3×ULN). Note: Transaminases can be up to 5×ULN in the setting of liver metastases.
9. Adequate coagulation parameters (defined as PT and/or PTT≤1.5×ULN or platelets ≥100,000).
10. Adequate neutrophils (defined as absolute neutrophil count ≥1,500/mm3).
11. A lesion that either: i. is intended to be accessed bronchoscopically, or ii. is intended to be accessed with CT guided transthoracic injection and in the estimation of the radiologist performing the procedure will not require transversing a bullae that significantly increases the risk of pneumothorax.
12. In women who have not experienced menopause, negative pregnancy test prior to initiation of treatment and adequate contraception throughout treatment. Adequate forms of contraception include: i. 2 adequate barrier methods; ii. a barrier method plus a hormonal method of contraception; iii. abstaining from sexual activity throughout the trial, starting with the screening visit through 120 days after the last dose of pembrolizumab.

Subjects excluded are those having previous systemic therapy for Stage IV NSCLC, including chemotherapy, radiation therapy or noncytotoxic investigational agents and patients with sensitizing EGFR mutations and/or ALK gene rearrangements.

### Materials and Methods:

Pembrolizumab: Anti-PD-1 immunotherapy pembrolizumab (Keytruda) is provided by Merck and available upon the initiation of the trial.

Recombinant human GM-CSF and IL-4: Recombinant human GM-CSF and human IL-4 is manufactured and tested for clinical use under the Current Good Manufacturing Practices (cGMP) guidelines by R&D SYSTEMS, Inc. (Minneapolis, Minn.).

cGMP-Ad-CCL21 Virus Derivative: Fully tested and quantified GMP-grade replication-deficient adenovirus expressing human CCL21 (cGMP-Ad-CCL21) is used. Also available is cGMP-Ad-CCL21 (Lot L0604006) manufactured by BDP/SAIC-Frederick, Inc. SAIC-Frederick, Inc. is a contractor to the National Cancer Institute (NCI-Frederick) (Frederick, Md.), which is a government-owned, contractor operated facility.

Ad-CCL21-tranduced DC: Ad-CCL21-tranduced DC (Ad-CCL21-DC) is prepared in UCLA Human Gene and Cell Therapy Facility (HGCTF) as described (45). Briefly, autologous human monocyte-derived DC is prepared from the patient's peripheral blood under GMP conditions and cultured in vitro with 800 U/ml of clinical-grade recombinant human GM-CSF and 400 U/ml of clinical-grade recombinant human IL-4. Following 6-7 days of culturing, DC are transduced with cGMP-grade Ad-CCL21 by ultra-centrifugation at 2000×g at 37° C. for 2 hours. After various biosafety tests, Ad-CCL21-modifed DC (Ad-CCL21-DC) is then prepared for injection into the patient's lung cancer by CT-guided or bronchoscopic delivery.

Collection of PBMCs: Patient's autologous leukapheresis product is collected 14 days prior to the first scheduled Ad-CCL21-DC administration. The leukapheresis procedure is performed in the Hemapheresis Unit. Samples from the leukapheresis product are sent for sterility testing. PBMCs are isolated from the leukapheresis product by density gradient centrifugation on Ficoll-Paque (GMP grade, GE Life Sciences). PBMCs are cryopreserved at a concentration of 1×108 cells/ml of DMSO containing freezing media (70% RPMI-1640 (Life Technologies)+20% autologous serum+10% DMSO in controlled rate freezing cryocontainers. The PBMCs are stored at −80° C. Samples of the PBMCs in DMSO containing freezing media will be sent for sterility testing.

6 days prior to the planned Ad-CCL21-DC administration to the patients (Protocol Day −6, +15, and +36), cryopreserved PBMC is rapidly thawed in a water bath at 37° C. The cells are washed in serum free RPMI-1640 medium and plated at 5-10×106 cells/ml (75-150×106 cells per 15 ml per T175 flask) in 5% culture media (RPMI-1640+5% autologous serum). After allowing adherence for 2 hours at 37° C., non-adherent cells are gently removed by washing with serum free RPMI-1640 media. Adherent cells are cultured in 5% culture media for 6 days in a 37° C., 5% CO2 incubator in the presence of rhGM-CSF (800 U/ml) and rhIL-4 (400 U/ml). All culture media is filtered through a 0.22-μm filter prior to use. After 4 days of DC culturing, an aliquot of supernatant from each flask is harvested for sterility. After 5 days of DC culturing, an aliquot of each flask's supernatant is pooled and sent for mycoplasma PCR testing.

After 6 days in culture, DCs are harvested from the T175 flasks by gently pipetting from culture, washed in serum free RPMI-1640, and re-suspended in 1 ml of serum free RPMI-1640. Prior to Ad-CCL21 transduction, a sample (˜1%) of the DC cell suspension is sent for DC phenotyping. Ad-CCL21 is added to the DC at 1167 VP/cell (Virus:DC MOI of 100:1). Preclinical characterization of the Ad-CCL21 demonstrated adenoviral transduction at MOI 100:1 yielded the highest levels of CCL21 production without compromising cell viability (83). The cell suspension is centrifuged at 2000×g at 25° C. for two hours (84).

Bronchoscopy and intratumoral injection of DC: Bronchoscopy is performed as an outpatient procedure. Patients will have complete screening laboratory work verifying that they have adequate renal function, liver function, white blood cell counts, platelet counts, PT/PTT, and pulmonary function. Patients will have fasted for at least 6 hours prior to the study. Patients are attached to continuous monitoring (ECG, non-invasive blood pressure monitoring, and pulse oximetry) and provided with oxygen at 2-6 liters/min via nasal cannula. To ablate the gag-reflex, patients are treated with a hand held nebulizer containing 3 ml of 2 or 4% lidocaine followed by topical spraying with 20% benzocaine or 4% lidocaine as needed. Due to their smoking histories, some patients may be treated with albuterol 2.5 mg and atrovent 1.0 mg by hand-held nebulizer as indicated by the clinical situation at the time of the procedure. Conscious sedation will be administered in accordance with institutional Conscious Sedation Guidelines using intravenous anxiolytics (e.g. midazolam in 0.5-1 mg aliquots) and analgesics (e.g. fentanyl 25 mg aliquots).

A fiberoptic video-bronchoscope will be introduced orally or transnasally by physicians trained and certified in the procedure. The larynx, trachea, and bilateral airways (mainstem, lobar, and segmental bronchi) will be systematically visualized. Two ml aliquots of 2% lidocaine will be applied to the bronchial mucosa at selected sites if additional topical anesthesia is required. The amount of lidocaine that is administered in this manner is recorded and will not exceed a 4 mg/kg or total dose of 280 mg, in order to prevent lidocaine toxicity.

Prior to injecting Ad-CCL21-DC, four needle biopsies will be performed using a 19-gauge Wang transbronchial needle (Mill-Rose laboratories, Inc., 15 mm needle length and working length 140 cm, diameter of 2 mm) to obtain a core biopsy of the tumor for in vitro monitoring studies. The mass will be visually divided into 4 quadrants and needle biopsies will be obtained from each of these quadrants. Patients will receive 4 injections of Ad-CCL21-DC, one in each quadrant, using a 22-gauge transbronchial cytology needle (13 mm needle length and 144 cm working length and diameter of 1.8 mm). The dead space of the cytology needle assembly will be carefully filled with the Ad-CCL21-DC suspension and 0.25 ml of cell suspension injected into each site. Prior to injecting Ad-CCL21-DC, gentle aspiration will be applied to ensure that the needle tip has not been placed into a vascular structure. Alternatively, instead of using the Wang transbronchial needle, biopsies may be obtained with the use of bronchoscopic biopsy forceps or by endobronchial ultrasound guided needle. If a patient has a complication such as pneumothorax or bleeding after the pre-treatment biopsy, the Ad-CCL21-DC will still be injected unless the risk is considered inappropriate by the study investigator and/or proceduralist. If the risk is determined to be too high for the patient to receive the injection of Ad-CCL21-DC, the patient will remain on pembrolizumab alone. Patients who cannot complete the entire Ad-CCL21-DC dose schedule will be excluded from the MTD/MAD determination, and will be replaced with another participant. The vaccine may be delivered by direct bronchoscopy or endobronchial ultrasound guidance.

CT-guided intratumoral injection of Ad-CCL21-DC: CT-guided procedures will be performed as an outpatient procedure. Patients will have complete screening laboratory work verifying that they have adequate renal function, liver function, white blood cell counts, platelet counts, PT/PTT, and pulmonary function. All CT scans will be performed on a GE High Speed Advantage scanner (GE Medical Systems, Milwaukee, Wis.). Limited axial scans will be performed through the target lesion at 3 mm, 5 mm, or 10 mm collimation, depending on the lesion size. Scans will be done before, during, and after needle placement to plan needle placement approach and confirm successful and uncomplicated injection. All patients will be prepped and draped in the usual sterile fashion. 1% lidocaine will be used to achieve local anesthesia. No sedation is planned. Patients will be monitored with respect to temperature, pulse, and blood pressure pre- and post-procedure. Oxygen saturation and blood pressure will be monitored throughout the procedure.

For lung tumors >3 cm in size, the mass will be divided into 4 equal quadrants, and each quadrant will undergo biopsy and Ad-CCL21-DC injection. For tumors <3 cm in size, the mass will undergo biopsy and injection at only a single site. A 19-gauge outer needle with a 20-gauge inner biopsy needle will be used to obtain a core biopsy specimen. Following insertion of the needle tip into a selected target lesion, aspiration will be applied to the syringe to confirm no entry into vascular structures. A core biopsy of the lung tumor will be obtained. A 22-gauge Chiba spinal needle inserted through the 19-gauge outer needle will be used to deliver Ad-CCL21-DC. A total volume of 1.2 ml of Ad-CCL21-DC will be injected equally into 4 separate quadrants or at a single site depending on the size of the tumor, followed by a 1 ml of normal saline flush administration. With all transpleural intrapulmonary injections, 2-5 ml of autologous blood will be administered along the parenchymal needle tract as a blood patch with withdrawal of the needle. If a pneumothorax occurs due to the transthoracic injection, when feasible the pneumothorax will be evacuated as the injection needle is withdrawn. In the case of a pneumothorax or bleeding after biopsies, Ad-CCL21-DC will still be injected unless the risk is considered inappropriate by the study investigator and/or proceduralist. If the risk is too high, the patient will remain on pembrolizumab alone. Patients who cannot complete the entire Ad-CCL21-DC dose schedule will be excluded from the MTD/MAD determination, and will be replaced with another participant.

### Trial Study

A phase I, non-randomized, dose escalating, multi-cohort trial followed by a dose expanding portion at the dose established during ExD is conducted. Patients with pathologically confirmed and radiographically measurable stage IV NSCLC expressing PD-L1 in less than 50% of cells, who have tumor accessible by CT-guided intervention or bronchoscopy, and who are naïve to systemic treatment for NSCLC will be selected for this study.

Up to 24 patients are enrolled. During dose escalation, up to 12 patients are evaluated, 3 or 6 patients at each dose level, depending on the presence or absence of a dose-limiting toxicity (DLT). During dose expansion, 12 patients are evaluated at the dose established during dose escalation (ExD). Ad-CCL21-DC will be delivered by bronchoscopic or CT-guided IT injection. Pembrolizumab is administered intravenously.

During dose escalation, a modified 3+3 design is used. Three patients are assigned to each cohort. Patients enrolled into a given cohort receive the same Ad-CCL21-DC dose by CT-guided or bronchoscopic IT injection followed by IV pembrolizumab 200 mg one hour after DC injection on days 0, 21, and 42, and IV pembrolizumab 200 mg every three weeks thereafter for up to a year. The Ad-CCL21-DC dose is 1×107 cells/injection in the first cohort (1), and is increased to 3×107 cells/injection (2) pending the tolerability in earlier cohort. Dose escalation may proceed only if all 3 patients enrolled in the lower dose cohort experience no DLT or 1 of 6 patients in a cohort has a DLT. If a patient dies within 30 days of receiving investigational treatment, the study will be held until further evaluation, and potential risks vs. benefits of continuing the study will be discussed with the UCLA JCCC DSMB. If the dose regimen in cohort 1 (Ad-CCL21-DC 1×107 cells/injection) is not well tolerated, de-escalation to Ad-CCL21-DC to 5×106 cells/injection will be allowed (˜1). If the dose regimen specified for Cohort (2) (Ad-CCL21-DC 3×107 cells/injection) is not the maximum tolerated dose (MTD), no further dose escalation will be conducted, and this dose level will be defined as maximum administered dose (MAD). Patients will be enrolled at UCLA Medical Center as out-patients.

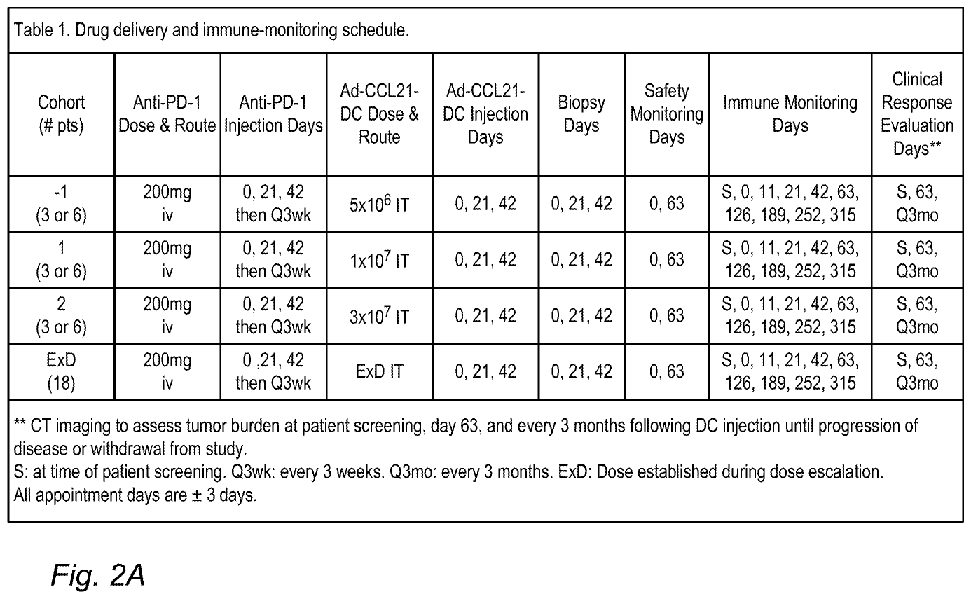

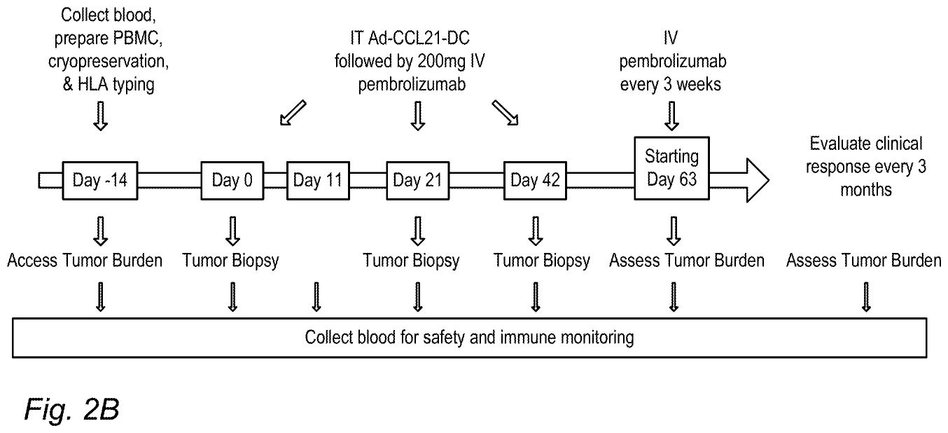

FIG. 2 sets out the proposed regimen and dose expansion. During dose escalation the study should establish maximum tolerated dose (MTD)/maximum administered dose (MAD) as the dose level below the one at which more than 1/6 patients experience dose-limiting toxicities (DLT), or Dose Cohort (2), if there is 0 or 1/6 DLT in the cohort. Dose Expansion is characterized by ORR defined by RECIST v1.1 criteria.

Dose limiting toxicity (DLT) is defined as grade 3 or greater toxicity as defined by the NCI Common Toxicity Criteria version 4.0. In a given cohort, if 0 of 3 patients have a DLT, then we will escalate to the next dose by enrolling patients into the next cohort. Dose escalation may proceed only if all 3 patients have been enrolled into a lower dose cohort and no DLT is seen over a 28-day period. If 2 or 3 of the 3 have a DLT, the dose will be de-escalated to the previous dose. If 1 of 3 has a DLT, 3 more patients will be enrolled into the same cohort and receive the same Ad Ad-CCL21-DC dose. If a total of 1 of 6 has a DLT, the dose will be escalated, and patients will be enrolled into the next cohort. If 2 or more of the 6 have a DLT, then the dose is de-escalated to the previous dose. If it is decided to de-escalate to a dose where 3 patients have already been treated, then an additional 3 patients are treated at that same dose. If 2 or more of the additional patients have a DLT, then we will de-escalate to the previous dose. In any case, if a patient dies within 30 days of receiving investigational treatment, the study will be held until further evaluation, and potential risks vs. benefits of continuing the study will be discussed with the UCLA JCCC DSMB. In determining the MTD/MAD, no more than 6 patients will be treated at a given dose. The MTD/MAD is defined as the dose level at which fewer than 2 of 6 patients experience a DLT. In the event that 2 or 3 of the 3 have a DLT in the first cohort (A), study accrual will be held. Serious adverse events (SAE) not related to study drug (for example pneumothorax from the biopsy or injections, or other forms of procedure related complications) will not be counted as DLT. Patients withdrawing therapy for reasons other than DLT will be replaced with other subjects until at least 3 evaluable patients have completed treatment in a given cohort.

Treatment Modification and General Management of Toxicities: For any Grade 1 toxicity, there will be no dose modification. If Grade 2 toxicity develops, the investigator may elect to continue the intratumoral treatment with careful monitoring, or to withhold the treatment until values return to Grade 1 or less, then restart the treatment. One of the expected grade 2 toxicities associated with lung tumor injections is the iatrogenic introduction of a pneumothorax. If a pneumothorax occurs, the patient may be monitored, or have the pneumothorax evacuated by needle thoracostomy. A recalcitrant or enlarging pneumothorax may necessitate chest tube placement. This is an expected complication, which it is not anticipated will necessitate halting further injections. Accordingly, if the pneumothorax resolves with thoracostomy evacuation within 72 hrs of the injection and is not radiographically apparent at the time of the next injection, the protocol will proceed as prescribed.

If Grade 3 toxicity is observed that is likely attributable to the biological effects of DC, further administration will be withheld until the toxicity is reduced to less than grade 2, and the investigator evaluates the subject to determine clinical acceptability for continuing the protocol. Treatment can resume at the discretion of the investigators, but with a reduction in the dose of intratumoral DC by 50%. If the observed grade 3 toxicity is an expected procedural complication (e.g.: a persistent air leak that has not responded to thoracostomy drainage at 7 days), intratumoral treatment will be withheld until the complication resolves. If the complication resolves between days 7-14 following the initial vaccine administration, and investigator evaluates the subject to be clinically acceptable for continuing with the protocol, then the vaccine administration will resume at full dose.

Delays of administration >2 weeks due to toxicity, or repeat Grade 3 toxicity despite dose reduction will result in subject discontinuation of the protocol. Subjects who have been discontinued due to delays of administration will be replaced until the necessary number of patients to be evaluated is met.

Any Grade 4 toxicity will result in removal of the subject from the protocol, continued observation and treatment of the subject as indicated by the clinical situation. The development of Grade 4 toxicity in any one subject in a cohort, thought to be related to the protocol treatment, will result in termination of treatment at that dosing level, and definition of the MTD/MAD at the dose administered in the prior cohort.

Any subject death within 30 days of study drug administration will result in the study to be held, until evaluation of the cause is determined. If the death is attributed to the protocol treatment, further treatment in all subjects will cease, and the study will be discontinued.

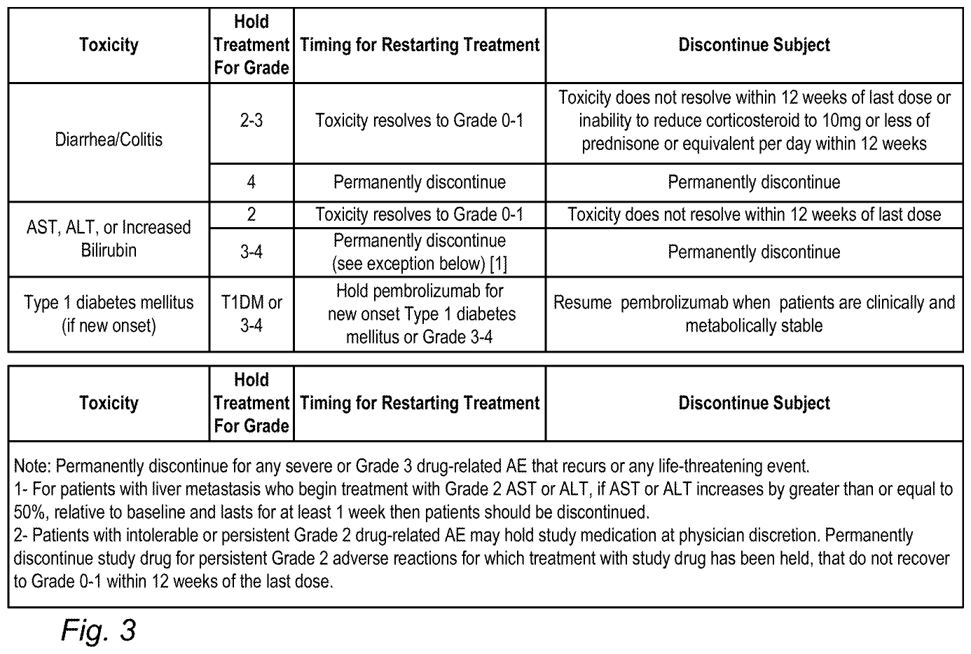

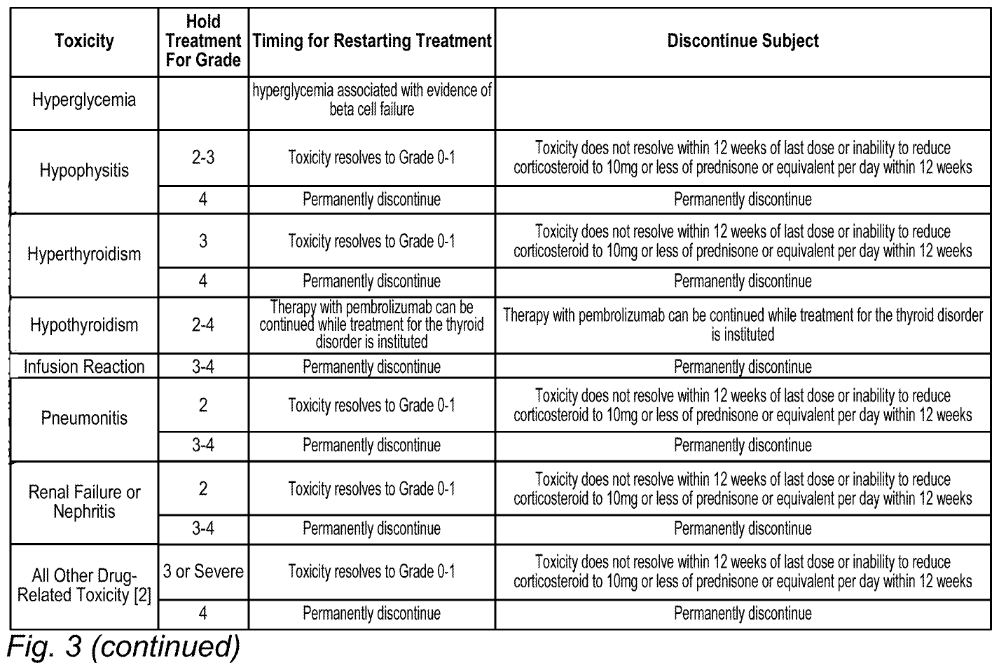

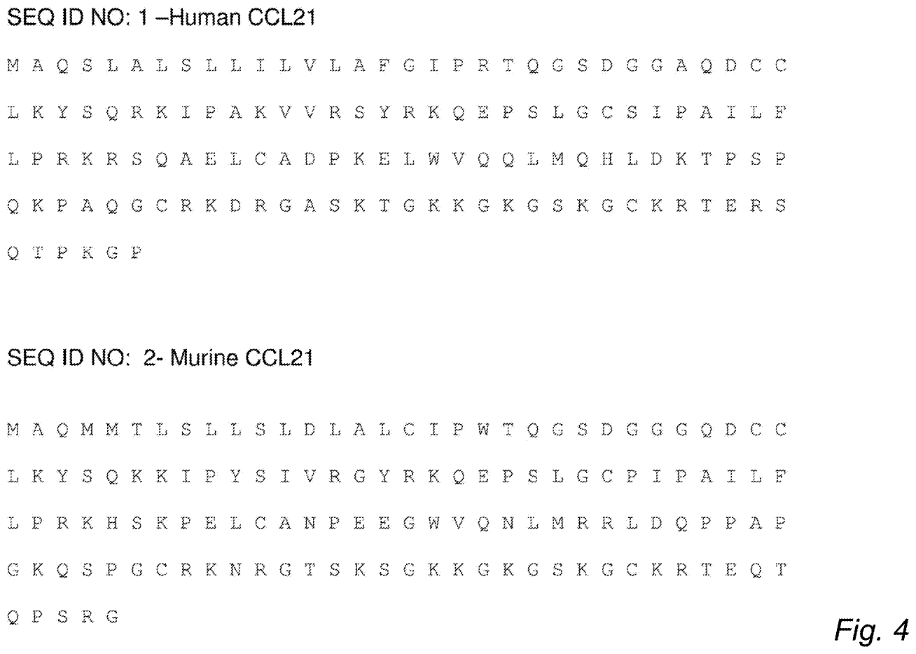

Adverse events (both non-serious and serious) associated with pembrolizumab exposure may represent an immunologic etiology. These adverse events may occur shortly after the first dose or several months after the last dose of treatment. Pembrolizumab must be withheld for drug-related toxicities and severe or life-threatening AEs as set out in FIG. 3.

Biological and clinical responses: All patients are monitored to assess the clinical efficacy of the treatment and to define potential determinants of the response. Clinical response will be evaluated by CT scans at screening, on day 63, and every 3 months thereafter until progression of disease or patient withdrawal from the study. Immune monitoring will be performed using tumor biopsies and peripheral blood samples as detailed below. Tumor biopsies are also utilized to determine the mutational load and tumor associated neoantigens.

Multiplex immunofluorescence (MIF) will be utilized to characterize the immune phenotype of the TME. Table 1 below lists surrogate markers of immune phenotypes (81), with the specific emphasis on CD8 and PD-L1 staining. These assays will be performed with biopsy samples collected on day 0, 21 and 42.

TABLE 1

| Panel 1 | Panel 2 |
|---|---|
| AE1/AE3 | AE1/AE3 |
| PD-L1 | PD-1 |
| CD4 | Granzyme B |
| CD8 | CD57 |
| CD3 | CD45RO |
| CD68 | FOXP3 |
| DAPI | DAPI |

Whole Exome Sequencing (WES) is performed using genomic DNA (gDNA) from pre- and post-treatment tumor samples to identify somatic mutational load and tumor-associated neoantigens. Patient's diagnostic tissue block (or paraffin-embedded day 0 biopsy if such block is not available) and paraffin-embedded day 42 biopsy is utilized as pre- and post-treatment samples, respectively. Tumor cells are enriched by laser capture microdissection, followed by gDNA isolation. Patient's gDNA from PBMC serves as a germline reference in the mutational analyses. These studies will elucidate the evolution of neoantigen landscape following the combination therapy.

Peripheral blood samples: Screening, days 0, 11, 21, 42, 63, 126, 189, 252, 315.

CD8+PD-1+T lymphocytes are sorted from PMBC (day 0, 21, 63, 126, 252) by flow cytometry and their gDNA isolated using a DNeasy kit (Qiagen). TCR-β chain CDR3 regions are amplified and deep sequencing performed with survey ImmunoSeq assay (Adaptive Biotechnologies) to determine clonality.

Mass cytometry (CyTOF) is used to perform comprehensive immunophenotyping of immune cell subsets from PBMCs (day 0, 11, 42, 189, 315) and elucidate the evolution of immune responses through the course of treatment.

As a complementary approach to MIF and CyTOF, Nanostring PanCancer Immune Profiling Panel is utilized to monitor the anti-tumor immune responses in the study patients. This panel consists of 770 genes and includes 109 genes related to cell surface markers for 24 different immune cell types, as well as over 500 genes that represent all categories of immune response, including key checkpoint blockade genes. It also includes 40 reference genes for data normalization in the expression analysis. To identify leukocyte subpopulations based on gene expression, the CYBERSORT method is utilized (82). Briefly, RNA is isolated from PBMCs (day 0, 11, 42, 189, 315), using a miRNeasy RNA Isolation Kit (Qiagen). RNA quality and integrity are assessed by running on an Agilent Bioanalyzer. Two hundred nanograms of RNA is used for the Nanostring analysis at the UCLA Center for Systems Biomedicine.

Quantification of anti-Ad IgM and IgG antibodies is performed by ELISA, using plasma from day 0, 63 blood samples. Antigen-specific ELISPOT assays to monitor immune responses to HLA-matched TAAs commonly seen in NSCLC patients is performed, using PBMC from day 0, 21, 63, 126, 252.

Clinical responses: Screening, day 63, Q3mo until progression of disease or withdrawal from study: CT scans are carried out for evaluation of tumor burden (irRECIST Criteria for patient management, RECIST 1.1 for final analysis). Toxicity by NCI Common Toxicity Criteria v 4.0 is used to define adverse event (AE) associated with the combination therapy of Ad-CCL21-DC and pembrolizumab.

Adverse events are monitored throughout the study in two phases. Phase one constitutes safety monitoring during the treatment, where ELISA for Adenovirus-specific IgM and IgG antibodies are performed with patient plasma, using the blood samples from day 0 and 63. The second phase occurs during clinical response evaluations, which is performed on screening day, day 63 and once every 3 months thereafter until progression of disease or patient withdrawal from the study. Toxicity by NCI Common Toxicity Criteria is applied to define AEs associated with the combination therapy of Ad-CCL21-DC and pembrolizumab. All AEs are monitored and reported to regulatory authorities and IRB/IECs in accordance with all applicable global laws and regulations.

Subjects have serial measurements of evaluable target lesions and non-target lesions that are graded according to both the immune related and standard RECIST v1.1 guidelines prior to Day 0, on Day 63, and every 3 months thereafter until disease progression or withdrawal from the protocol:
Target Lesions
Complete Response (CR): Disappearance of all target lesions
Partial Response (PR): At least a 30% decrease in the sum of the longest diameter (LD) of target lesions, taking as reference the baseline sum LD
Progressive Disease (PD): At least a 20% increase in the sum of the LD of target lesions, taking as reference the smallest sum LD recorded since the treatment started or the appearance of one or more new lesions.
Stable Disease (SD): Neither sufficient shrinkage to qualify for PR nor sufficient increase to qualify for PD, taking as reference the smallest sum LD since the treatment started.
Non-target Lesions
Complete Response (CR): Disappearance of all non-target lesions and normalization of tumor marker level.
Incomplete Response/Stable Disease (SD): Persistence of one or more nontarget lesion(s) or/and maintenance of tumor marker level above the normal limits.
Progressive Disease (PD): Appearance of one or more new lesions and/or unequivocal progression of existing non-target lesion. Although a clear progression of “non-target” lesions only is exceptional, in such circumstances, the opinion of the treating physician should prevail, and the progression status should be confirmed later on by the review panel (or study chair).

During dose escalation, the primary endpoint is to establish a maximum tolerated dose (MTD)/maximum administered dose (MAD). Because the combination therapy proposed herein has not been tested in prior clinical trials, this endpoint is appropriate to determine the most potent dose which can be safely given. During dose expansion, the primary endpoint is to define the objective response rate of the Ad-CCL21-DC/pembrolizumab combination therapy. Particularly, the study is interested in determining whether Ad-CCL21-DC administration augments PD-1/PD-L1 inhibition in the control of cancer progression. Throughout the study, secondary endpoints include defining toxicities using CTCAE v4.0 to grade adverse events (AE) and determining effects of treatment on drug target activity by comprehensive immune monitoring studies.

### Example 3. Efficacy of Combination Therapy in a High Mutational Burden Model

Studies described here demonstrate the efficacy of the combination therapy on tumors with high mutational burden.

Recent studies demonstrate that human NSCLC (over 85% of lung cancers) possesses high nonsynonymous mutational burden, a biomarker associated with clinical benefit of PD-1 blockade. KRAS mutations are the most prevalent oncogenic driver in NSCLC (˜25% in LUAC), and recent studies reveal that co-occurring mutations in the STK11/LKB1 (KL) and TP53 (KP) tumor suppressor genes define distinct subgroups of NSCLC with unique TME immune profiles. LKB1 mutation has recently been identified as a major driver of primary resistance to PD-1 blockade in KRAS-mutant lung adenocarcinoma (LUAC).

Genetically engineered mouse models (GEMMs) of lung cancer, such as KrasG12D (LKR13), KrasG12D;P53−/−(KP) and KrasG12D;P53−/−;Lkb1−/− (KPL), possess common driver mutations of human NSCLC. However, recent studies and our data reveal low single nucleotide variants (SNVs) in these models that do not mimic human NSCLC, which frequently possess high tumor mutational burden (TMB). Therefore, we developed GEMMs bearing various mutational loads by in vitro exposure of LKR13, KP, and KPL cells to tobacco carcinogen methyl-nitrosourea (MNU) to recapitulate the mutational landscape of human NSCLC. Whole exome sequencing (WES) of these mutant cell lines revealed significant increases in mutational loads and intratumoral heterogeneity.

Studies were conducted to evaluate the efficacy of IT CCL21-DC and IP anti-PD-1 combination therapy in two of the newly established GEMMs of lung cancer harboring low (KPL) and high (KPL-3M; KPL with three in vitro MNU exposures) mutational loads. IT administration of CCL21-DC significantly potentiated anti-PD-1 efficacy in KPL model, while CCL21-DC or anti-PD-1 alone did not show significant effect (FIGS. 5A and 5B). In KPL-3M model, CCL21-DC and anti-PD-1 monotherapies elicit moderate efficacy, while the combination therapy resulted in effective tumor eradication (FIGS. 5C and 5D). These data indicate that IT CCL21-DC delivery improves the efficacy of anti-PD-1 for lung cancer treatment and in particular, efficacy in a high tumor burden model.

FIG. 5 A-D shows that intratumoral (IT) administration of CCL21-DC potentiates anti-PD-1 efficacy in genetically engineered mouse models (GEMMs) of lung cancer. A) IT CCL21-DC and anti-PD-1 combination in murine KrasG12D;P53−/−;Lkb−/− (KPL) model. FVB mice were subcutaneously inoculated with 7.5×104 KPL cells. On day 7, mice bearing <25 mm3 tumors were treated with a) vehicle control; b) IT CCL21-DC (106 CCL21-DC/dose on day 7, 11, 15); c) IP anti-PD-1 (200 μg/dose on day 7, 9, 11, 13,15); d) combination of IT CCL21-DC and IP anti-PD-1 at the same time points as above. Tumor volume was recorded. B) Same as in A except that tumor weight at the end of the study was presented. C) Same as in A except that KPL-3M cells (1.0×105 cells), which bear high mutational loads were utilized. D) Same as in B except that KPL-3M cells were utilized. P values were determined by non-paired t-test. n.s., not significant; *, P<0.05; **, P<0.005; ****, P<0.00005.

### Example 4. Phase I Trial of CCL21 and Anti-PD-1 Antibody to Treat Non-Small Cell Lung Cancer Having High Mutational Burden

A trial is carried out as described in detail in Example 2 above, wherein patients entered into the trial are prescreened and enrolled if tumors have a high mutational burden, determined for example by the methods described herein such as but not limited to diagnostic assays from FoundationOne (Cambridge, Mass.), such as FoundationOne CDx™ FoundationOne®, FoundationAct®, or FoundationOne®Heme. Other methods may be used to determine that tumors have a high mutational burden. This trial will then determine the safety and efficacy of intratumoral (IT) injection of CCL21 gene-modified DC (Ad-CCL21-DC) when combined with intravenous pembrolizumab in patients with previously untreated, advanced NSCLC, whose tumors express PD-L1 in less than 50% of tumor cells, and having tumors with high mutational burden.

### REFERENCES

1. L. A. Torre, R. L. Siegel, A. Jemal, Adv Exp Med Biol 893, 1-19 (2016).
2. Y. Tang et al., Onco Targets Ther 10, 4527-4534 (2017).
3. N. A. M. Howlader N., Krapcho M., Miller D., Bishop K., Altekruse S. F., Kosary C. L., Yu M., Ruhl J., Tatalovich Z., Mariotto A., Lewis D. R., Chen H. S., Feuer E. J., Cronin K. A., National Cancer Institute. Bethesda, Md. (2016).
4. R. L. Siegel, K. D. Miller, A. Jemal, C A Cancer J Clin 65, 5-29 (January-February, 2015).
5. M. Reck et al., N Engl J Med 375, 1823-1833 (Nov. 10, 2016).
6. A. T. Shaw et al., J Clin Oncol 27, 4247-53 (Sep. 10, 2009).
7. T. J. Lynch et al., N Engl J Med 350, 2129-39 (May 20, 2004).
8. J. G. Paez et al., Science 304, 1497-500 (Jun. 4, 2004).
9. W. Pao et al., Proc Natl Acad Sci USA 101, 13306-11 (Sep. 7, 2004).
10. J. P. Koivunen et al., Clin Cancer Res 14, 4275-83 (Jul. 1, 2008).
11. R. Chiarle et al., Nat Rev Cancer 8, 11-23 (January, 2008).
12. E. B. Garon et al., N Engl J Med 372, 2018-28 (May 21, 2015).
13. G. V. Scagliotti et al., J Clin Oncol 26, 3543-51 (Jul. 20, 2008).
14. J. Brahmer et al., N Engl J Med 373, 123-35 (Jul. 9, 2015).
15. H. Borghaei et al., N Engl J Med 373, 1627-39 (Oct. 22, 2015).
16. L. Horn et al., J Clin Oncol, JCO2017743062 (Oct. 12, 2017).
17. R. S. Herbst et al., Lancet 387, 1540-50 (Apr. 9, 2016).
18. A. Rittmeyer et al., Lancet 389, 255-265 (Jan. 21, 2017).
19. D. P. Carbone et al., N Engl J Med 376, 2415-2426 (Jun. 22, 2017).
20. C. J. Langer et al., Lancet Oncol 17, 1497-1508 (November, 2016).
21. M. D. Hellmann et al., Lancet Oncol 18, 31-41 (January, 2017).
22. J. M. Lee et al., Clin Cancer Res 23, 4556-68 (May 3, 2017).
23. S. Novello et al., Ann Oncol 27, v1-v27 (September, 2016).
24. M. L. Spiegel et al., Cancer (Nov. 10, 2017).
25. S. N. Gettinger et al., J Clin Oncol 33, 2004-12 (Apr. 20, 2015).
26. J. R. Brahmer et al., 2014 ASCO Meeting, J Clin Oncol 32:5s, 2014 (suppl: abstr 8021).
27. V. K. Anagnostou, J. R. Brahmer, Clin Cancer Res 21, 976-84 (Mar. 1, 2015).
28. R. S. Herbst et al., Nature 515, 563-7 (Nov. 27, 2014).
29. P. C. Tumeh et al., Nature 515, 568-71 (Nov. 27, 2014).
30. L. Robert et al., Oncoimmunology 3, e29244 (2014).
31. I. Yoshino et al., Cancer Res 54, 3387-90 (Jul. 1, 1994).
32. S. M. Dubinett et al., Am J Respir Cell Mol Biol 22, 524-7 (May, 2000).
33. N. P. Restifo et al., J Exp Med 177, 265-72 (Feb. 1, 1993).
34. S. Sharma et al., Journal of Immunology 163, 5020-8 (Nov. 1, 1999).
35. M. K. Srivastava et al., Immunotherapy 4, 291-304 (March, 2012).
36. L. X. Zhu et al., J Immunol 170, 3585-91 (Apr. 1, 2003).
37. S. Sharma et al., Cancer Res 65, 5211-20 (Jun. 15, 2005).
38. F. Baratelli et al., Am J Transl Res 2, 356-67 (2010).
39. S. M. Dubinett, J. M. Lee, S. Sharma, J. J. Mule, Cancer J 16, 325-35 (July-August, 2010).
40. V. W. Chan et al., Blood 93, 3610-6 (Jun. 1, 1999).
41. J. G. Cyster, J Exp Med 189, 447-50 (Feb. 1, 1999).
42. J. F. Arthur et al., Cancer Gene Ther 4, 17-25 (January-February, 1997).
43. P. Gallo et al., Gene Ther 12 Suppl 1, S84-91 (October, 2005).
44. L. H. Butterfield, L. Vujanovic, Curr Opin Investig Drugs 11, 1399-408 (December, 2010).
45. F. Baratelli et al., J Transl Med 6, 38 (Jul. 22, 2008). CLIN2-10784 CLIN 2: Clinical Trial Stage Projects 65
46. S. C. Yang et al., Cancer Res 66, 3205-13 (Mar. 15, 2006).
47. S. C. Yang et al., Clin Cancer Res 10, 2891-901 (Apr. 15, 2004).
48. T. Okazaki, A. Maeda, H. Nishimura, T. Kurosaki, T. Honjo, Proc Natl Acad Sci USA 98, 13866-71 (Nov. 20, 2001).
49. J. L. Riley, Immunol Rev 229, 114-25 (May, 2009).
50. K. A. Sheppard et al., FEBS Lett 574, 37-41 (Sep. 10, 2004).
51. M. E. Keir, M. J. Butte, G. J. Freeman, A. H. Sharpe, Annu Rev Immunol 26, 677-704 (2008).
52. J. Banchereau, R. M. Steinman, Nature 392, 245-52 (Mar. 19, 1998).
53. R. M. Steinman, Annu Rev Immunol 30, 1-22 (2012).
54. E. W. Roberts et al., Cancer Cell 30, 324-336 (Aug. 8, 2016).
55. S. Spranger, D. Dai, B. Horton, T. F. Gajewski, Cancer Cell 31, 711-723 e4 (May 8, 2017).
56. J. Constantino et al., Immunol Res 65, 798-810 (August, 2017).
57. A. D. Garg et al., Oncoimmunology 6, e1328341 (2017).
58. V. Cerundolo, I. F. Hermans, M. Salio, Nat Immunol 5, 7-10 (January, 2004).
59. R. L. Sabado, S. Balan, N. Bhardwaj, Cell Res 27, 74-95 (January, 2016).
60. N. Nagarsheth, M. S. Wicha, W. Zou, Nat Rev Immunol 17, 559-572 (September, 2017).
61. A. P. Vicari et al., J Immunol 165, 1992-2000 (Aug. 15, 2000).
62. S. Sharma et al., J Immunol 164, 4558-63 (May 1, 2000).
63. S. Sharma et al., Mol Cancer 2, 22 (Apr. 15, 2003).
64. S. Sharma et al., Cancer Res 61, 6406-12 (Sep. 1, 2001).
65. A. Ribas et al., Cancer Res 60, 2218-24 (Apr. 15, 2000).
66. S. Hillinger et al., Journal of Immunology 171, 6457-65. (2003).
67. S. M. Dubinett et al., J Natl Cancer Inst 87, 593-7 (Apr. 19, 1995).
68. A. Hagenbaugh et al., J Exp Med 185, 2101-10 (Jun. 16, 1997).
69. M. Huang et al., Cancer Res 58, 1208-16 (Mar. 15, 1998).
70. S. M. Dubinett et al., Pathobiology 59, 372-7 (1991).
71. S. M. Dubinett, J. T. Kurnick, R. L. Kradin, Am J Respir Cell Mol Biol 1, 361-9 (November, 1989).
72. J. Kim et al., J Immunol 155, 2240-7 (Aug. 15, 1995).
73. W. H. McBride et al., Cancer Res 52, 3931-7 (Jul. 15, 1992).
74. S. M. Dubinett et al., Cancer Immunol Immunother 36, 156-62 (1993).
75. J. M. Lee et al., in Journal for ImmunoTherapy of Cancer. (2014), vol. 2, pp. 020.
76. Y. Carmi et al., Nature 521, 99-104 (May 7, 2015).
77. N. A. Rizvi et al., Science 348, 124-8 (Apr. 3, 2015).
78. K. Politi, R. S. Herbst, Clin Cancer Res 21, 2213-20 (May 15, 2015).
79. P. Sharma, S. Hu-Lieskovan, J. A. Wargo, A. Ribas, Cell 168, 707-723 (Feb. 9, 2017).
80. W. Zou, J. D. Wolchok, L. Chen, Sci Transl Med 8, 328rv4 (Mar. 2, 2016).
81. E. R. Parra et al., Sci Rep 7, 13380 (Oct. 17, 2017).
82. A. M. Newman et al., Nat Methods 12, 453-7 (May, 2015).
83. K. Riedl et al., Mol Cancer 2, 35 (Nov. 2, 2003).
84. N. Nishimura, Y. Nishioka, T. Shinohara, S. Sone, Hum Gene Ther 12, 333-46 (Mar. 1, 2001).
85. E. Guenova et al., Eur J Immunol 38, 3138-49 (November, 2008).
86. G. Trinchieri, Blood 84, 4008-27 (Dec. 15, 1994).
87. S. E. Macatonia et al., J Immunol 154, 5071-9 (May 15, 1995).
88. G. Breton et al., J Exp Med 213, 2861-2870 (Dec. 12, 2016).
89. M. Guilliams et al., Immunity 45, 669-684 (Sep. 20, 2016).
90. M. Alcantara-Hernandez et al., Immunity 47, 1037-1050 e6 (Dec. 19, 2017).
91. M. Dauer et al., J Immunol 170, 4069-76 (Apr. 15, 2003).
92. W. Zou, L. Chen, Nat Rev Immunol 8, 467-77 (June, 2008).
93. J. Galon, W. H. Fridman, F. Pages, Cancer Res 67, 1883-6 (Mar. 1, 2007).

1. A method of treating cancer or a solid tumor having a high mutational burden in a subject comprising
a. administering to the subject (i) a SLC polypeptide, (ii) a polynucleotide encoding the SLC polypeptide, (iii) a cell comprising the polynucleotide encoding the SLC polypeptide, or (iv) any combination thereof, and
b. administering to the subject an immune checkpoint inhibitor.

2. The method of claim 1, wherein the immune checkpoint inhibitor is selected from the group consisting of a CTLA-4 inhibitor, a CTLA-4 receptor inhibitor, a PD-1 inhibitor, a PD-L1 inhibitor, a PD1-L2 inhibitor, a 4-1BB inhibitor, an OX40 inhibitor, a LAG-3 inhibitor, a TIM-3 inhibitor, or a combination thereof.

3. The method of claim 1, wherein the immune checkpoint inhibitor is an antibody, optionally, a monoclonal antibody.

4. The method of claim 1, wherein the immune checkpoint inhibitor is a CTLA-4 inhibitor, optionally, ipilimumab or tremilimumab.

5. The method of claim 1, wherein the immune checkpoint inhibitor is a PDl inhibitor selected from a group consisting of: Nivolumab, Pembrolizumab, Pidilizumab, Lambrolizumab, BMS-936559, Atezolizumab, and AMP-224, AMP224, AUNP12, BGB108, MCLA134, MEDIO680, PDROOl, REGN2810, SHR1210, STIAllOX, STIAlllO and TSR042.

6. The method of claim 1, wherein the immune checkpoint inhibitor is a PD-L1 inhibitor selected from a group consisting of: BMS-936559, MPDL3280A, MEDI-4736, MSB0010718C, ALN-PDL, BGBA317, KD033, KY1003, STIA100X, STIA1010, STIA1011, STIA1012 and STIA1014.

7. The method of claim 1, wherein the SLC polypeptide comprises an amino acid sequence of SEQ ID NO: 1 or SEQ ID NO: 2.

8. The method of claim 1, wherein the polynucleotide encoding the SLC polypeptide is inserted into a vector and the vector is administered to the subject.

9. The method of claim 8, wherein the vector is an adenoviral vector.

10. The method of claim 9, wherein the adenoviral vector is a replication-deficient adenoviral vector.

11. The method of claim 1, wherein the cell comprising the polynucleotide encoding the SLC polypeptide is an antigen presenting cell (APC).

12. The method of claim 11, wherein the APC is a dendritic cell.

13. The method of claim 12, wherein the dendritic cell is autologous to the subject.

14-15. (canceled)

16. The method of claim 1, wherein the subject comprises a solid tumor and the cells are administered to the subject intratumorally.

17. The method of claim 1, wherein the solid tumor is a non-small cell lung carcinoma (NSCLC) solid tumor.

18-23. (canceled)

24. The method of claim 1,
identifying the presence of a high mutational burden in the tumor of the subject prior to steps (b) and (c).

25-54. (canceled)

55. The method of claim 83, wherein the cancer is a high mutational burden cancer.

56. The method of claim 55 wherein the anti-PD-1 antibody is selected from the group consisting of Nivolumab, Pembrolizumab, Pidilizumab, and Lambrolizumab.

57-82. (canceled)

83. A method for treating cancer or reducing the recurrence of cancer in a subject in need thereof comprising administering an effective amount of a combination therapy comprising a) dendritic cells comprising a vector-CCL21 (vector-CCL21-DC) construct on days 0, 21, and 42, and b) an effective amount of anti-PD-1 antibody every three weeks starting on day 0, optionally wherein the vector is an adenoviral vector (Ad-CCL21-DC).

84. The method of claim 83 wherein the anti-PD-1 antibody is selected from the group consisting of Nivolumab, Pembrolizumab, Pidilizumab, and Lambrolizumab.

85-102. (canceled)


SEQUENCE LISTING (2 sequences)

SEQ ID NO: 1 - protein, 134 aa, Homo sapiens
MAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 2 - protein, 133 aa, Mus musculus
MAQMMTLSLLSLDLALCIPWTQGSDGGGQDCCLKYSQKKIPYSIVRGYRKQEPSLGCPIPAILFLPRKHSKPELCANPEEGWVQNLMRRLDQPPAPGKQSPGCRKNRGTSKSGKKGKGSKGCKRTEQTQPSRG